(19) United States
(12) Patent Application Publication
(10) Pub. No.: US 2023/0241050 A1
(43) Pub. Date: Aug. 3, 2023

(54) APPLICATIONS OF HPK1 KINASE INHIBITOR IN PREVENTING AND/OR TREATING ANIMAL PATHOGEN INFECTION

(71) Applicant: ZHUHAI YUFAN BIOTECHNOLOGIES CO., LTD, Zhuhai, Guangdong (CN)

(72) Inventors: Xingyu LIN, Zhuhai, Guangdong (CN); Tingting LU, Zhuhai, Guangdong (CN)

(21) Appl. No.: 18/009,954

(22) PCT Filed: May 27, 2021
(86) PCT No.: PCT/CN2021/096276

§ 371 Date: Dec. 12, 2022

(30) Foreign Application Priority Data
Jun. 16, 2020 (CN) 202010546816.X

(51) Int. Cl. A61K 31/4545; A61P 31/14; A61K 31/496; A61K 31/497; A61K 31/444; A61K 31/4439; A61K 31/551; A61K 31/5377; A61K 31/501; A61K 31/506; A61P 29/00

(52) U.S. Cl. CPC A61K 31/4545; A61K 31/444; A61K 31/496; A61K 31/497; A61K 31/501; A61K 31/506; A61K 31/551; A61K 31/4439; A61K 31/5377; A61P 29/00; A61P 31/14

(57) ABSTRACT

Use of an HPK1 kinase inhibitor in preventing and/or treating pathogen infection in animals. The HPK1 kinase inhibitor is a small molecule HPK1 kinase inhibitor, especially the compound of general formula I or a pharmaceutically acceptable salt, a stereoisomer, an ester, a prodrug, a solvate, or a deuterated compound thereof. The HPK1 kinase inhibitor can effectively treat diseases (such as feline infectious peritonitis) caused by infection of pathogens (specifically viruses, such as coronaviruses) in animals, improve animal survival, and provide enhanced commercial value and application prospects in the field of animal antiviral (specifically viruses, such as coronaviruses) medicaments.

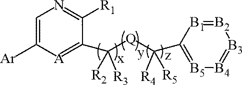

### TECHNICAL FIELD

The present invention relates to the technical field of chemical pharmaceutics, and in particular to use of an HPK1 kinase inhibitor in preventing and/or treating pathogen (specifically viruses, such as coronaviruses) infection in animals.

### BACKGROUND

Viral diseases are a group of diseases doing most serious harm to animals in veterinary practice, and particularly, the prevalence of some highly pathogenic viral diseases often cause enormous economic and mental losses to the breeders. Viral diseases in animals usually spread quickly, feature high morbidity rate and mortality rate, and seriously endanger the health and life of animals. Moreover, some viruses can be transmitted to human beings through animals, threatening the safety of human beings.

Viral diseases in animals can be caused by a wide variety of viruses, for example, viruses being of Herpesviridae, Rhabdoviridae, Reoviridae, Poxviridae, Asfarviridae, Adenoviridae, Parvoviridae, Circoviridae, Orthomyxoviridae, Paramyxoviridae and Coronaviridae. Coronaviruses are a large family of viruses widely present in the nature and are named for their morphological similarity to crown under an electron microscope. They mainly cause respiratory diseases and can infect various mammals, such as pigs, cattle, cats, dogs, minks and camels, and various birds. For example, porcine deltacoronavirus (PDCoV) is a novel porcine enteric coronavirus that can cause diarrhea and vomiting, rapid dehydration and death of organ failure in piglets of 5-15 days old, with morbidity and mortality rates up to 50% to 100%; avian infectious bronchitis is an acute highly contagious respiratory infectious diseases in chicken caused by infectious bronchitis virus and is clinically characterized by dyspnea, rales, cough, mouth breathing and sneezing, a non-nephropathogenic strain that does not cause complication generally leads to a low mortality, but to reduced egg production and quality in laying hens; bovine coronavirus (BCV) is a pathogenic virus of cattle, is generally regarded as an important pathogen of neonatal calf diarrhea at present, and can cause respiratory tract infection in cattle and winter bloody dysentery in adult cattle; the epidemic diarrhea (coronavirus enteritis) in minks, foxes and raccoon dogs is caused by coronavirus except for mink parvovirus enteritis, and infected dogs, foxes, raccoon dogs and minks usually demonstrate hemorrhagic gastroenteritis symptoms and are susceptible to cluster epidemic diarrhea, showing a high spreading speed, a mortality more than 30% and a higher morbidity in animals giving birth in the year is higher than in stud animals.

Although many vaccines against viral diseases have been developed with the development of pharmaceutical technology, virus mutants impose a burden of efficacy evaluation of the vaccines against the emerging viruses as well as a financial burden, and the morbidity and mortality of viral diseases still remain high, leaving a great demand for drugs with good antiviral efficacy to the field of veterinary.

### SUMMARY

The present invention provides use of an HPK1 kinase inhibitor in the preparation of a medicament for preventing and/or treating a disease or condition caused by or associated with pathogen infection in an animal.

In one embodiment of the present invention, the HPK1 kinase inhibitor described above is a small molecule inhibitor.

Specifically, the small molecule HPK1 kinase inhibitor described above is a compound of the following general formula I, or a pharmaceutically acceptable salt, a stereoisomer, an ester, a prodrug, a solvate or a deuterated compound thereof:

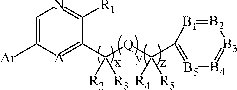

wherein,
A is selected from C and N;
Ar is selected from aromatic five-membered heterocyclic group, aromatic six-membered heterocyclic group and phenyl, wherein the aromatic five-membered heterocyclic group may be selected from: furyl, thienyl, pyrrolyl, pyrazolyl, imidazolyl, oxazolyl, thiazolyl and selenothiazolyl, and the aromatic six-membered heterocyclic group may be selected from: pyridyl, pyridazinyl, pyrimidinyl and pyrazinyl; optionally, H on the aromatic five-membered heterocyclic group, the aromatic six-membered heterocyclic group or the phenyl described above may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, -N heterocycloalkyl, —N heterocycloaryl, —O heterocycloaryl or —S heterocycloaryl, and wherein the alkyl moieties may be optionally substituted with one or more of the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, -N(C₀₋₁₀ alkyl)(C₀₋₁₀ alkyl), -OC₀₋₁₀ alkyl, —N heterocycloaryl, —O heterocycloaryl and —S heterocycloaryl;
R₁ is selected from: —H, halogen, —NO₂, —CN, C₁₋₅ linear/branched alkyl, C₃₋₁₀ cycloalkyl, -N(C₀₋₁₀ alkyl)(C₀₋₁₀ alkyl), —CF₃, —OCF₃, —OCHF₂, —OCH₂F and -OC₀₋₁₀ alkyl;
R₂ and R₃ are independently selected from: —H, halogen and C₁₋₁₀ linear/branched alkyl;
R₄ and R₅ are independently selected from: —H, deuterium, halogen, C₁₋₁₀ linear/branched alkyl and deuterated C₁₋₁₀ linear/branched alkyl;
Q is selected from O and S;
x and z are independently selected from integers between 0 and 6 (for example, 0, 1, 2, 3, 4, 5 or 6); y is 0 or 1;
B₁, B₂, B₃, B₄ and B₅ are independently selected from CR₆ and N; wherein each R₆ is independently selected from: —H, halogen, —CN, -OC₀₋₁₀ alkyl, C₁₋₁₀ linear/branched alkyl, heteroalkyl containing O or N, -N(C₀₋₁₀ alkyl)(C₀₋₁₀ alkyl), C₃₋₁₀ cycloalkyl, —C≡C—R₇, —O heterocycloalkyl, -N heterocycloalkyl, -COO(C₀₋₁₀ alkyl)(C₀₋₁₀ alkyl), -CO(C₀₋₁₀ alkyl)(C₀₋₁₀ alkyl), -CON(C₀₋₁₀ alkyl)(C₀₋₁₀ alkyl), and -N(C₀₋₁₀ alkyl)CO(C₀₋₁₀ alkyl)(C₀₋₁₀ alkyl), or two R₆, together with carbon atoms therebetween, form C₃₋₈ cycloalkyl or C₃₋₈ heterocycloalkyl containing —O— or —S—, —N heterocycloaryl, —O heterocycloaryl, —S heterocycloaryl or phenyl, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, —N heterocycloalkyl, —N heterocycloaryl, —O heterocycloaryl and —S heterocycloaryl;
R₇ is selected from H, C₁₋₅ linear/branched alkyl, C₃₋₁₀ cycloalkyl and

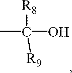

wherein R₈ and R₉ are independently selected from: —H, —CF₃, —CHF₂H, —CH₂F, C₁₋₁₀ linear/branched alkyl, -CH=C(C₀₋₁₀ alkyl)(C₀₋₁₀ alkyl), -C≡C(C₀₋₁₀ alkyl), C₃₋₁₀ cycloalkyl, -CO(C₀₋₁₀ alkyl)(C₀₋₁₀ alkyl), -CO(C₀₋₁₀ alkyl)(C₀₋₁₀ alkyl), -CON(C₀₋₁₀ alkyl)(C₀₋₁₀ alkyl), -N(C₀₋₁₀ alkyl)CO(C₀₋₁₀ alkyl)(C₀₋₁₀ alkyl), aromatic five-membered cyclic group and aromatic six-membered cyclic group, or R₈ and R₉, together with carbon atoms between R₈ and R₉, form C₃₋₈ cycloalkyl or C₃₋₈ heterocycloalkyl containing —O— or —S—, C₄₋₉ fused cycloalkyl, C₅₋₁₀ spiro cycloalkyl, C₄₋₉ bridged cycloalkyl, C₃₋₇ cyclolactam, C₃₋₇ cyclic lactone or C₃₋₇ cyclic ketone, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, —N heterocycloalkyl, —N heterocycloaryl, —O heterocycloaryl and —S heterocycloaryl, wherein the alkyl moieties may be optionally substituted with one or more of the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, -N(C₀₋₁₀ alkyl)(C₀₋₁₀ alkyl), -OC₀₋₁₀ alkyl, —N heterocycloaryl, —O heterocycloaryl and —S heterocycloaryl.

In one embodiment of the present invention, Q described above is O.

In one embodiment of the present invention, y described above is 1.

In one embodiment of the present invention, x described above is 0.

In one embodiment of the present invention, z described above is 1.

In one embodiment of the present invention, the compound described above has the following structure:

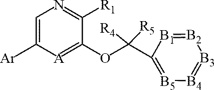

In one embodiment of the present invention, A described above is C.

In one embodiment of the present invention, the compound described above has the following structure:

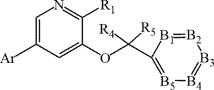

Specifically, R₄ and R₅ described above are independently selected from: —H and C₁₋₁₀ linear/branched alkyl; and more specifically, R₄ and R₅ are independently selected from: —H and C₁₋₃ linear/branched alkyl.

In one embodiment of the present invention, R₄ described above is H or —CH₃.

In one embodiment of the present invention, R₅ described above is H or —CH₃.

Specifically, at least one H on the aromatic five-membered heterocyclic group, the aromatic six-membered heterocyclic group or the phenyl described above is substituted with the following groups selected from: —SO₂, —SO₂NH₂, —NHSO₂, -CONH(C₀₋₁₀ alkyl), halogen, —CN, —OCF₃, —O heterocycloalkyl, —N heterocycloalkyl, C₁₋₁₀ linear/branched alkyl, C₃₋₁₀ cycloalkyl, -OC₀₋₁₀ alkyl, and -N(C₀₋₁₀ alkyl)(C₀₋₁₀ alkyl); and more specifically, at least one H on the aromatic five-membered heterocyclic group, the aromatic six-membered heterocyclic group or the phenyl described above is substituted with —O heterocycloalkyl or —N heterocycloalkyl.

Specifically, Ar described above is selected from: thiazolyl, selenothiazolyl, imidazolyl, pyrazolyl and pyridyl.

In one embodiment of the present invention, Ar described above is selected from:

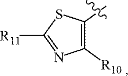

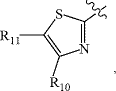

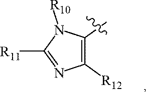

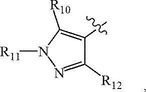

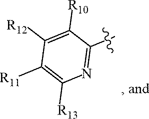

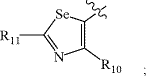

wherein R₁₀, R₁₂ and R₁₃ are independently selected from: —H, C₁₋₁₀ linear/branched alkyl, -N(C₀₋₁₀ alkyl)(C₀₋₁₀ alkyl), -OC₀₋₁₀ alkyl and C₃₋₁₀ cycloalkyl;
R₁₁ is selected from: —H, —O heterocycloalkyl, —N heterocycloalkyl, C₁₋₁₀ linear/branched alkyl, C₃₋₁₀ cycloalkyl, -OC₀₋₁₀ alkyl, -N(C₀₋₁₀ alkyl)(C₀₋₁₀ alkyl), -SO₂(C₀₋₁₀ alkyl), -O(C₀₋₁₀ alkyl), —O—phenyl, -S(C₀₋₁₀ alkyl), —N heterocycloaryl, —O heterocycloaryl and —S heterocycloaryl, wherein H on the C atom or heteroatom may be substituted with C₁₋₃ linear alkyl, -N(C₀₋₁₀ alkyl)(C₀₋₁₀ alkyl) or —CF₃.

Specifically, R₁₀, R₁₂ and R₁₃ described above may be independently selected from: —H, C₁₋₅ linear/branched alkyl and -N(C₀₋₁₀ alkyl)(C₀₋₁₀ alkyl); and more specifically, R₁₀, R₁₂ and R₁₃ may be independently selected from: —H, —CH₃, —CH₂CH₃ and —NH₂.

Specifically, R₁₁ described above may be selected from: —O heterocycloalkyl, —N heterocycloalkyl, -SO₂(C₀₋₃ alkyl), —O—phenyl, -S(C₀₋₄ alkyl), C₃₋₆ cycloalkyl and C₃₋₅ linear/branched alkyl, wherein H on the C atom or heteroatom may be substituted with —CH₃, —NH₂ or —CF₃; and more specifically, R₁₁ described above may be selected from:

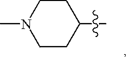

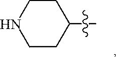

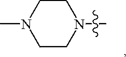

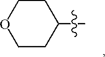

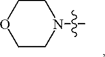

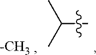

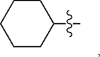

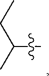

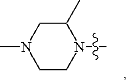

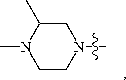

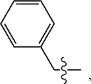

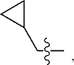

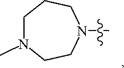

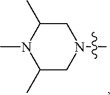

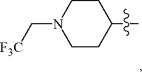

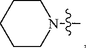

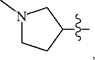

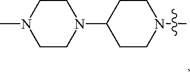

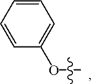

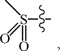

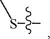

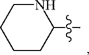

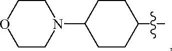

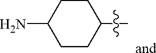

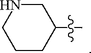

When R₁₀, R₁₂ and R₁₃ are adjacent to R₁₁, R₁₀, R₁₂ or R₁₃ and R₁₁, together with carbon atoms therebetween, can form C₃₋₈ cycloalkyl or C₃₋₈ heterocycloalkyl containing —O— or —S—, —N heterocycloaryl, —O heterocycloaryl, —S heterocycloaryl or phenyl.

In one embodiment of the present invention, the compound described above has the following structure:

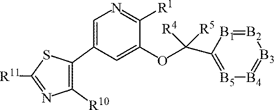

Specifically, R₁ described above may be selected from: —H, halogen, —NO₂, —CN, C₁₋₅ linear/branched alkyl, C₃₋₁₀ cycloalkyl, -N(C₀₋₁₀ alkyl)(C₀₋₁₀ alkyl), —CF₃, —OCF₃, —OCHF₂, —OCH₂F and -OC₀₋₁₀ alkyl; more specifically, R₁ described above may be selected from: —NO₂, -N(C₀₋₁₀ alkyl)(C₀₋₁₀ alkyl), -OC₀₋₁₀ alkyl and —OCF₃; and in one embodiment of the present invention, R₁ described above is —NH₂ or —NO₂.

In another embodiment of the present invention, at least one of B₁, B₂, B₃, B₄ and B₅ described above is N.

In one embodiment of the present invention, B₂ described above is CR₆, and at least one of B₁, B₃, B₄ and B₅ is N.

In one embodiment of the present invention, B₂ described above is CR₆, and B₁ is N.

In one embodiment of the present invention, B₂ described above is CR₆, and B₃ is N.

In one embodiment of the present invention, B₂ described above is CR₆, and B₄ is N.

In one embodiment of the present invention, B₂ described above is CR₆, and B₅ is N.

In one embodiment of the present invention, B₂ described above is CR₆, B₃ and B₄ are N, or B₃ and B₅ are N.

In one embodiment of the present invention, B₁, B₂, B₃, B₄ and B₅ described above are all CR₆, and in this case,

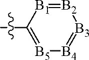

can be

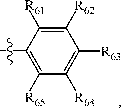

wherein R₆₁, R₆₂, R₆₃, R₆₄ and R₆₅ each have the definition as described above for R₆ in the present invention, and specifically, R₆₁, R₆₂, R₆₃, R₆₄ and R₆₅ may be independently selected from: —H, halogen, —CN, -OC₀₋₁₀ alkyl, C₁₋₁₀ linear/branched alkyl, heteroalkyl containing O or N, -N(C₀₋₁₀ alkyl)(C₀₋₁₀ alkyl), C₃₋₁₀ cycloalkyl, —C≡C—R₇, -O heterocycloalkyl and —N heterocycloalkyl, or R₆₁ and R₆₂, R₆₂ and R₆₃, R₆₃ and R₆₄, R₆₄ and R₆₅, together with carbon atoms therebetween, form C₃₋₈ cycloalkyl or C₃₋₈ heterocycloalkyl containing —O— or —S—, —N heterocycloaryl, —O heterocycloaryl, —S heterocycloaryl or phenyl, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, —N heterocycloalkyl, —N heterocycloaryl, —O heterocycloaryl and -S heterocycloaryl; wherein R₇ has the definition as described above in the present invention.

Specifically, R₆₃ described above may be selected from: —H, halogen, -OC₀₋₁₀ alkyl, C₁₋₁₀ linear/branched alkyl, -N(C₀₋₁₀ alkyl)(C₀₋₁₀ alkyl) and C₃₋₁₀ cycloalkyl; more specifically, R₆₃ may be selected from: —H, halogen, -OC₀₋₁₀ alkyl and C₁₋₁₀ linear/branched alkyl; and even more specifically, R₆₃ may be selected from: —H, —F and —OCH₃.

Specifically, R₆₂ described above may be selected from: —H, halogen, -OC₀₋₁₀ alkyl, —CN, C₃₋₁₀ cycloalkyl and —C≡C—R₇; more specifically, R₆₂ may be selected from: —H, —F, —Cl, —OCH₃, —CN,

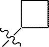

and -C≡C-R₇; and in one embodiment of the present invention, R₆₂ is C≡C-R₇.

Specifically, R₆₁, R₆₄ and R₆₅ described above are independently selected from: —H, halogen, —CN, -OC₀₋₁₀ alkyl, C₁₋₁₀ linear/branched alkyl, -N(C₀₋₁₀ alkyl)(C₀₋₁₀ alkyl), C₃₋₁₀ cycloalkyl, -C≡C-R₇, —O heterocycloalkyl, —N heterocycloalkyl, and C₁₋₅ linear/branched alkyl containing O or N, or R₆₄ and R₆₅, together with carbon atoms attached to R₆₄ and R₆₅, form C₃₋₈ cycloalkyl or C₃₋₈ heterocycloalkyl containing —O— or —S—, wherein H on the C atom may be substituted with —F; more specifically, R₆₁, R₆₄ and R₆₅ described above are independently selected from: —H, halogen, C₁₋₃ linear/branched alkyl, -OC₀₋₃ alkyl, and C₁₋₃ linear/branched alkyl containing N, or R₆₄ and R₆₅, together with carbon atoms attached to R₆₄ and R₆₅, form C₃₋₈ cycloalkyl or C₃₋₈ heterocycloalkyl containing —O—, wherein H on the C atom may be substituted with —F; and even more specifically, R₆₁, R₆₄ and R₆₅ described above are independently selected from: —H, —F, —Cl, —CH₃, —CH₂NH₂, —CN and —OCH₃, or R₆₄ and R₆₅, together with carbon atoms attached to R₆₄ and R₆₅, form five-membered cycloalkyl containing —O—. In one embodiment of the present invention, R₆₁, R₆₄ and R₆₅ described above are all H.

Specifically, R₇ described above is selected from: H, C₁₋₅ linear/branched alkyl, C₃₋₁₀ cycloalkyl and

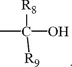

In one embodiment of the present invention, R₆₂ described above is

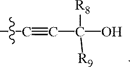

Specifically, R₈ and R₉ described above are independently selected from: —H, —CF₃, —CHF₂H, —CH₂F, C₁₋₁₀ linear/branched alkyl, -CH=C(C₀₋₁₀ alkyl)(C₀₋₁₀ alkyl), -C≡C(C₀₋₁₀ alkyl), C₃₋₁₀ cycloalkyl, aromatic five-membered cyclic group and aromatic six-membered cyclic group, or R₈ and R₉, together with carbon atoms between R₈ and R₉, form C₃₋₈ cycloalkyl or C₃₋₈ heterocycloalkyl containing —O or —S, C₄₋₉ fused cycloalkyl, C₅₋₁₀ spiro cycloalkyl, C₄₋₉ bridged cycloalkyl, C₃₋₇ cyclolactam, C₃₋₇ cyclic lactone, or C₃₋₇ cyclic ketone, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, -N heterocycloalkyl, —N heterocycloaryl, —O heterocycloaryl and —S heterocycloaryl, wherein the alkyl moieties may be optionally substituted with one or more of the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, -N(C₀₋₁₀ alkyl)(C₀₋₁₀ alkyl), -OC₀₋₁₀ alkyl, —N heterocycloaryl, —O heterocycloaryl and —S heterocycloaryl.

More specifically, R₈ and R₉ described above are independently selected from: —H, —CF₃, —CHF₂H, —CH₂F, C₁₋₁₀ linear/branched alkyl, -CH=C(C₀₋₁₀ alkyl)(C₀₋₁₀ alkyl), C₃₋₁₀ cycloalkyl and aromatic six-membered cyclic group, or R₈ and R₉, together with carbon atoms between R₈ and R₉, form C₃₋₈ cycloalkyl, C₄₋₇ fused cycloalkyl, C₅₋₉ spiro cycloalkyl, C₄₋₉ bridged cycloalkyl, C₃₋₇ cyclolactam, C₃₋₇ cyclic lactone, or C₃₋₇ cyclic ketone, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, —N heterocycloalkyl, —N heterocycloaryl, —O heterocycloaryl and —S heterocycloaryl, wherein the alkyl moieties may be optionally substituted with one or more of the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, -N(C₀₋₁₀ alkyl)(C₀₋₁₀ alkyl), -OC₀₋₁₀ alkyl, —N heterocycloaryl, —O heterocycloaryl and —S heterocycloaryl.

Even more specifically, R₈ and R₉ described above are independently selected from: —H, —CF₃, —CHF₂H, —CH₂F, C₁₋₅ linear/branched alkyl, -CH=CH(C₀₋₁₀ alkyl), C₃₋₁₀ cycloalkyl and aromatic six-membered cyclic group, or R₈ and R₉, together with carbon atoms between R₈ and R₉, form C₃₋₆ cycloalkyl, C₄₋₆ fused cycloalkyl, C₅₋₈ spiro cycloalkyl, C₄₋₈ bridged cycloalkyl, C₃₋₇ cyclolactam, C₃₋₇ cyclic lactone, or C₃₋₇ cyclic ketone, wherein H on the C atom may be substituted with the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, C₁₋₁₀ linear/branched alkyl, -N(C₀₋₁₀ alkyl)(C₀₋₁₀ alkyl), -OC₀₋₁₀ alkyl, C₃₋₁₀ cycloalkyl, —O heterocycloalkyl, —N heterocycloalkyl, —N heterocycloaryl, —O heterocycloaryl and —S heterocycloaryl, wherein the alkyl moieties may be optionally substituted with one or more of the following groups: —SO₂, -SO₂N(C₀₋₁₀ alkyl)(C₀₋₁₀ alkyl), -N(C₀₋₁₀ alkyl)SO₂(C₀₋₁₀ alkyl), -CON(C₀₋₁₀ alkyl)(C₀₋₁₀ alkyl), -N(C₀₋₁₀ alkyl)CO(C₀₋₁₀ alkyl), -N(C₀₋₁₀ alkyl)COO(C₀₋₁₀ alkyl), -OCON(C₀₋₁₀ alkyl)(C₀₋₁₀ alkyl), halogen, —CN, —OCH₂F, —OCHF₂, —OCF₃, -N(C₀₋₁₀ alkyl)(C₀₋₁₀ alkyl), -OC₀₋₁₀ alkyl, —N heterocycloaryl, —O heterocycloaryl and —S heterocycloaryl. In one embodiment of the present invention, R₈ and R₉ described above are independently selected from: —H, —CF₃, —CHF₂, —CH₂F, —CH₃, —CH₂CH₃, —CH═CH₂,

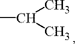

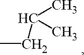

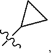

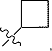

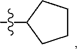

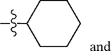

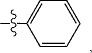

or R₈ and R₉, together with carbon atoms between R₈ and R₉, form

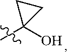

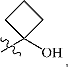

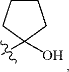

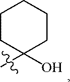

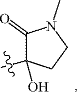

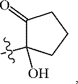

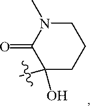

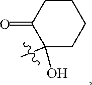

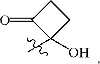

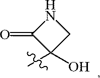

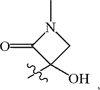

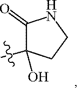

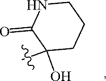

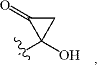

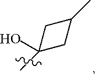

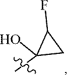

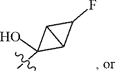

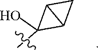

In one embodiment of the present invention, the compound described above has the following structure:

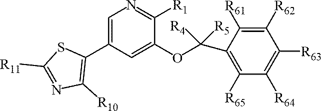

Specifically, the compound described above in the present invention may have the following structures:

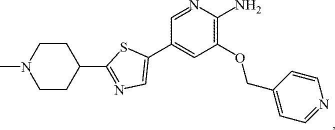

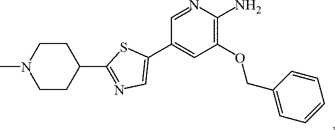

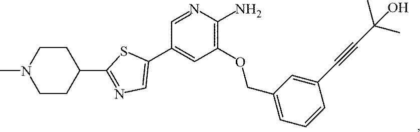

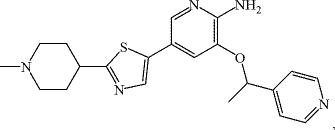

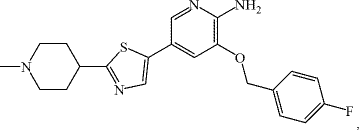

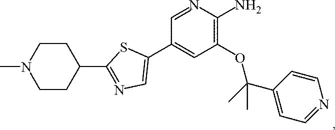

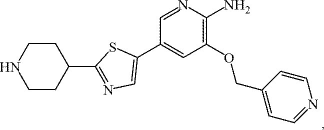

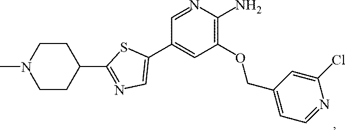

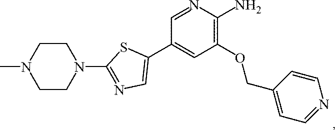

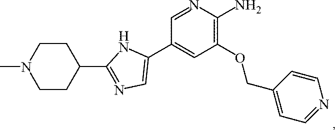

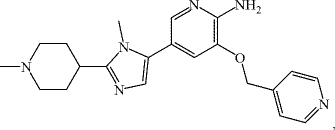

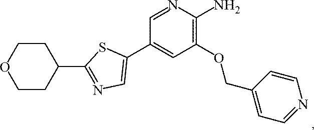

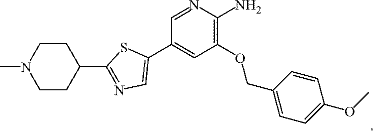

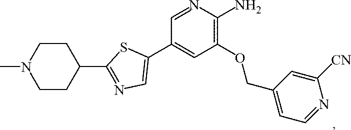

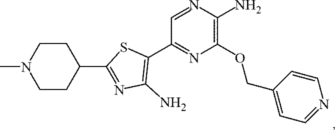

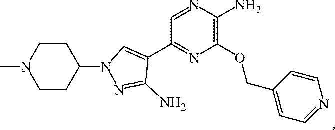

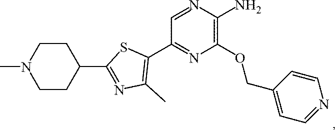

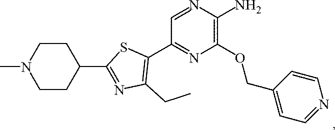

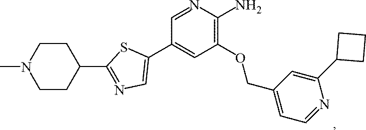

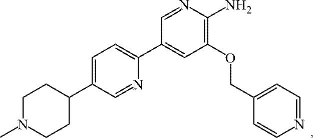

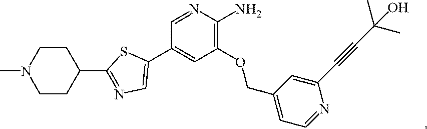

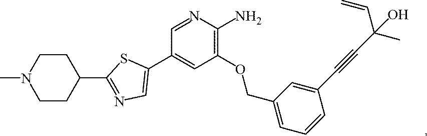

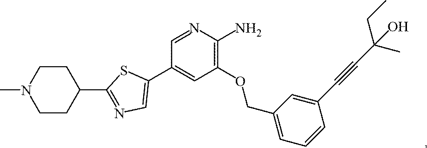

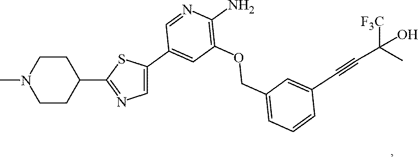

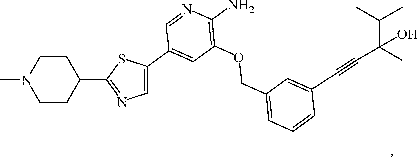

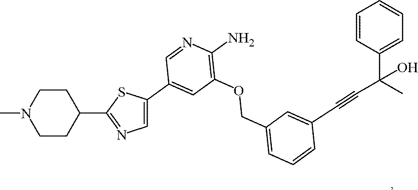

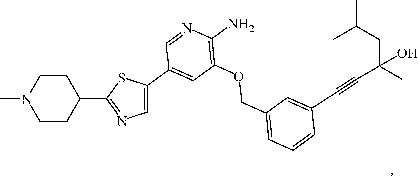

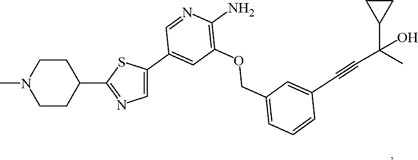

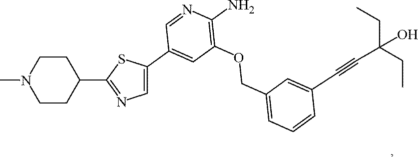

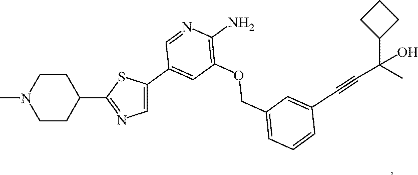

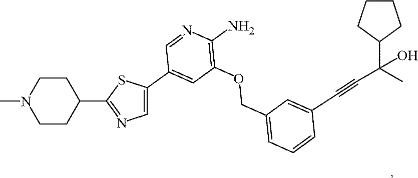

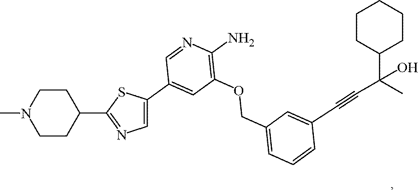

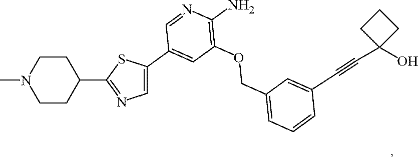

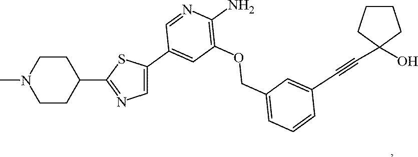

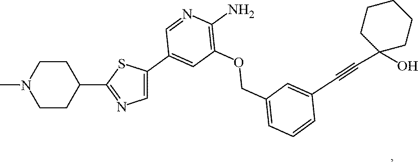

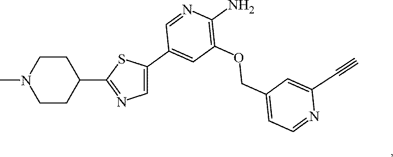

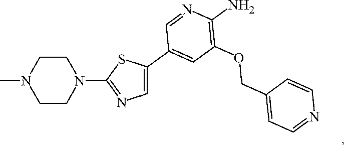

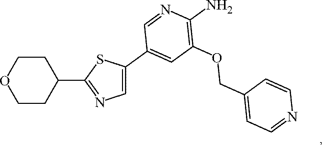

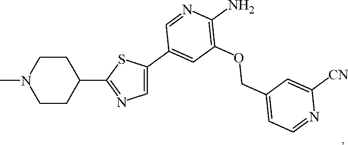

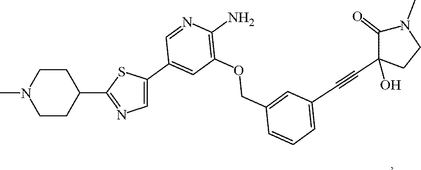

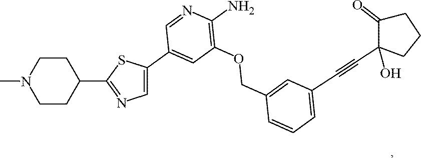

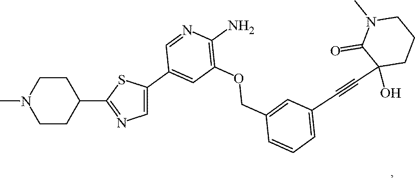

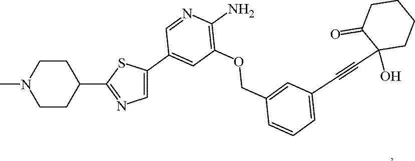

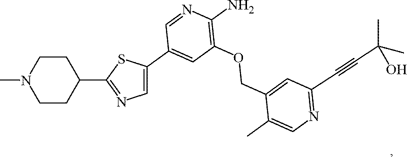

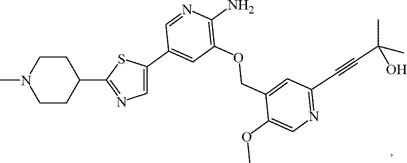

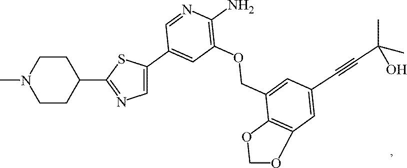

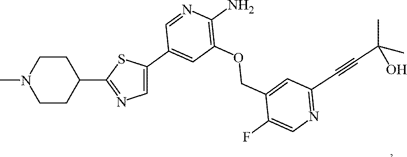

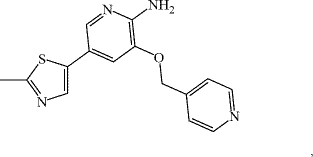

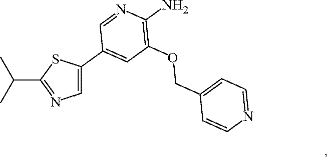

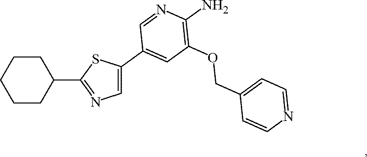

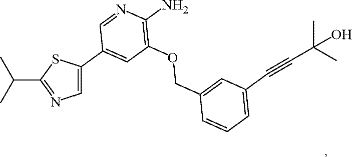

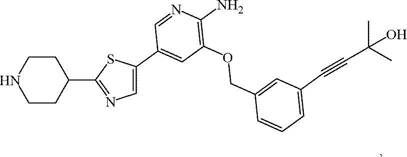

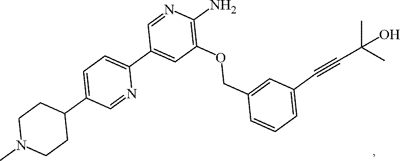

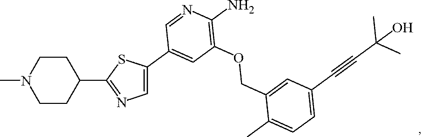

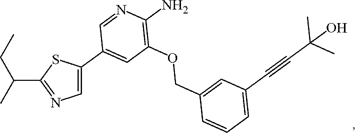

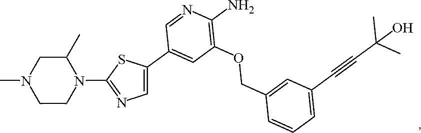

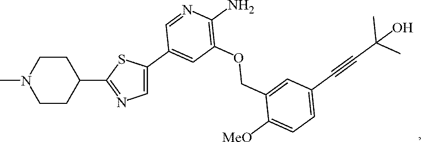

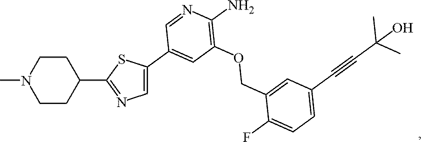

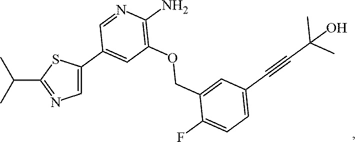

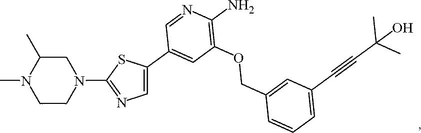

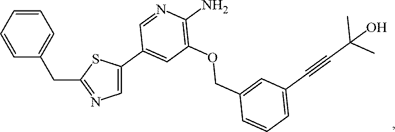

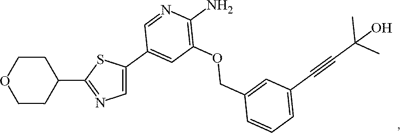

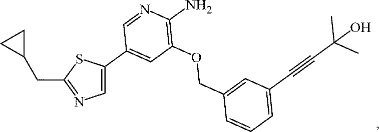

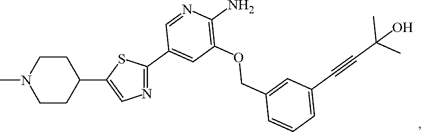

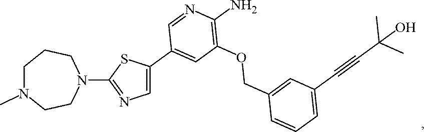

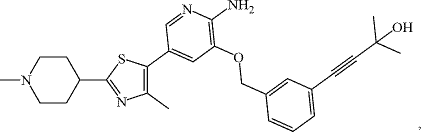

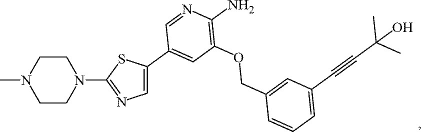

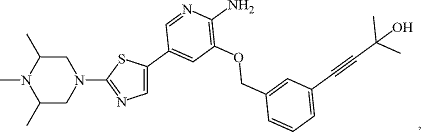

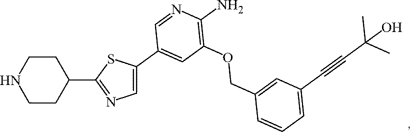

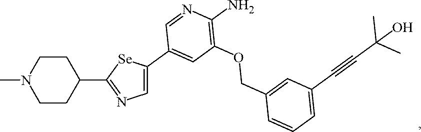

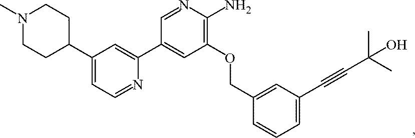

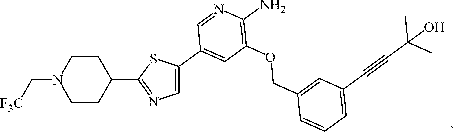

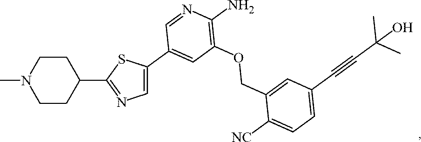

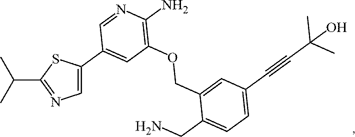

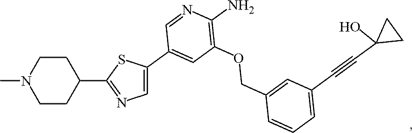

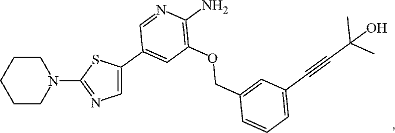

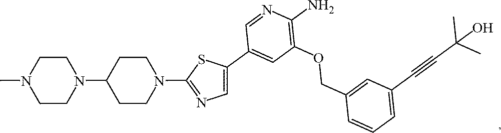

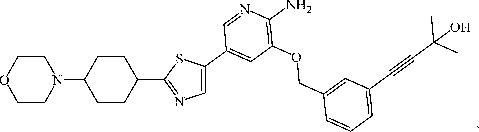

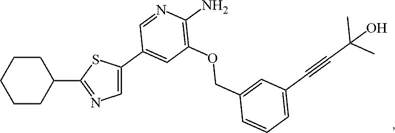

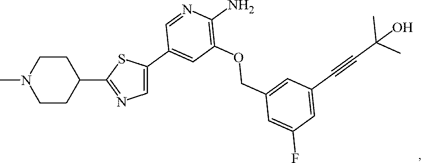

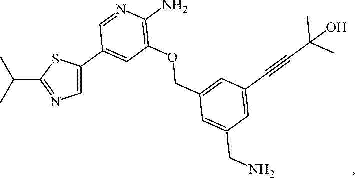

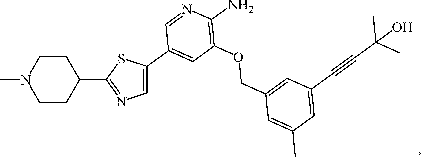

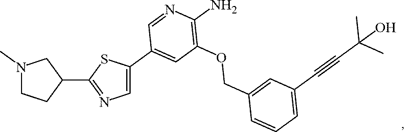

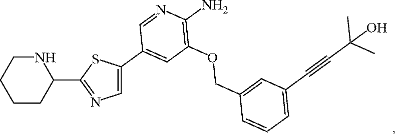

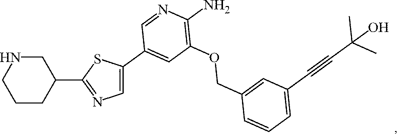

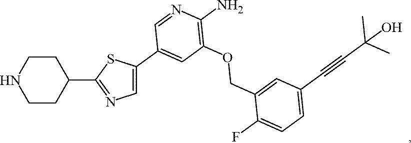

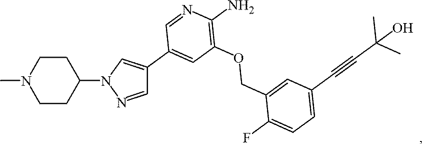

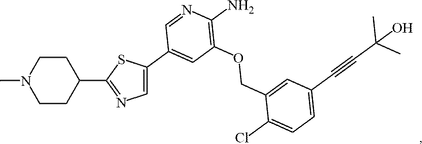

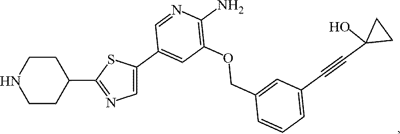

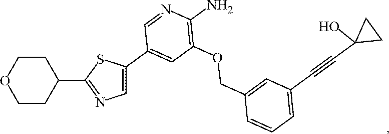

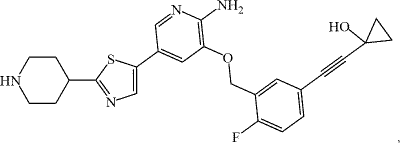

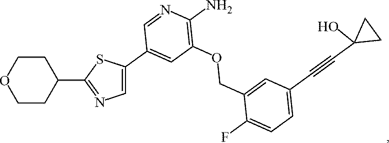

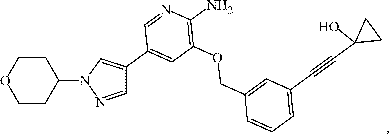

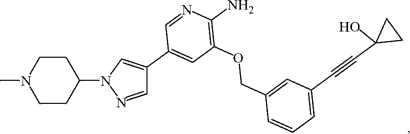

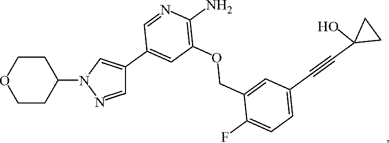

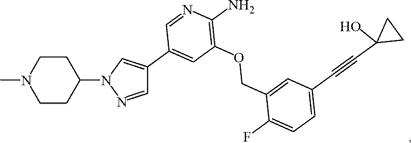

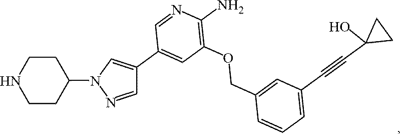

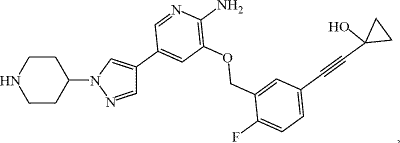

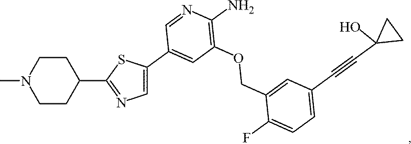

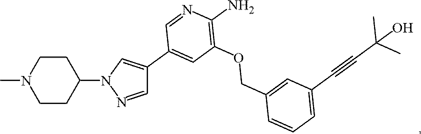

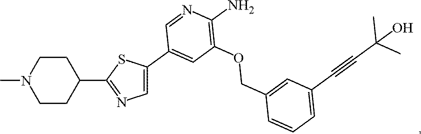

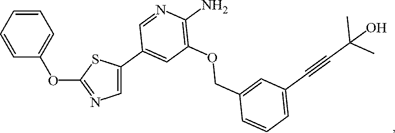

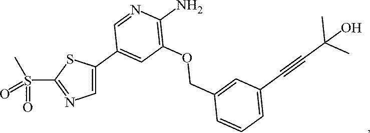

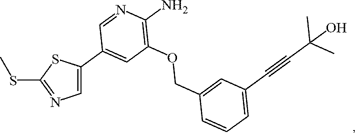

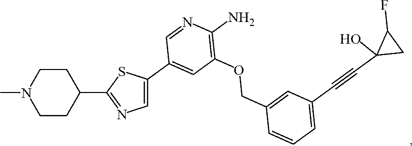

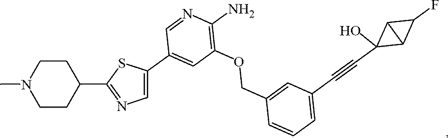

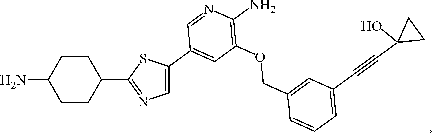

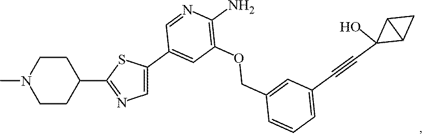

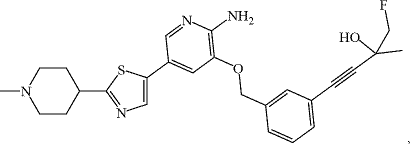

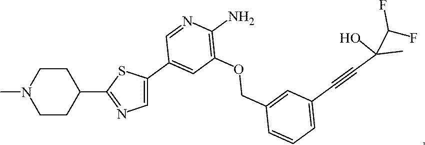

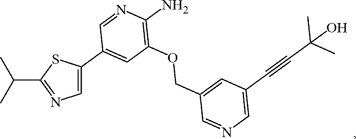

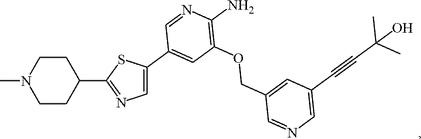

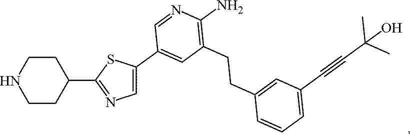

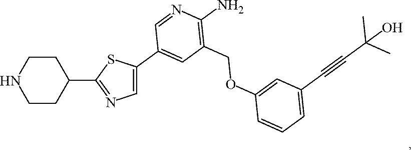

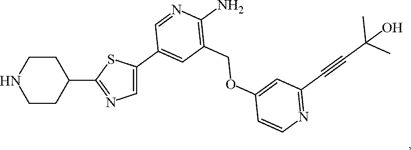

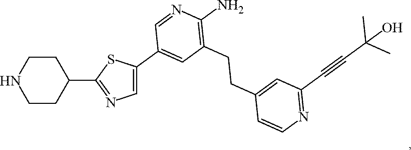

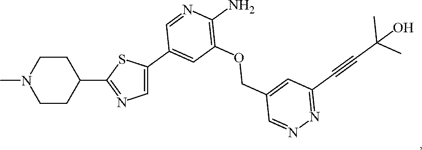

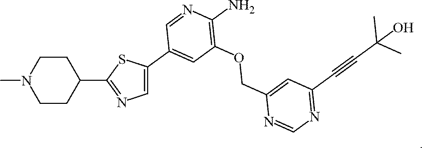

In one embodiment of the present invention, the compound described above has the following structure:

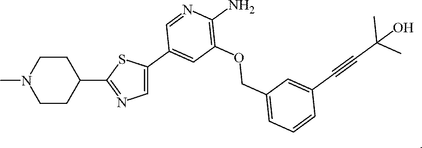

The pharmaceutically acceptable salts of the present invention include acid addition salts and base addition salts.

Specifically, the acid addition salts described above include, but are not limited to, salts derived from inorganic acids, such as hydrochloric acid, nitric acid, phosphoric acid, sulfuric acid, hydrobromic acid, hydroiodic acid and phosphonic acid, and salts derived from organic acids, such as aliphatic mono-carboxylic acid and aliphatic dicarboxylic acid, phenyl-substituted alkanoic acid, hydroxyalkanoic acid, alkanedioic acid, aromatic acid, aliphatic sulfonic acid and aromatic sulfonic acid. Thus, these salts include, but are not limited to, sulfate, pyrosulfate, bisulfate, sulfite, bisulfite, nitrate, phosphate, monohydrogen phosphate, dihydrogen phosphate, metaphosphate, pyrophosphate, hydrochloride, hydrobromide, iodate, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, tosylate, phenylacetate, citrate, lactate, tartrate, and methanesulfonate, as well as salts comprising amino acids such as arginate, gluconate and galacturonate. The acid addition salts described above can be prepared by contacting the free base form with a sufficient amount of the desired acid to form the salt in a conventional manner. The free base form can be regenerated by contacting the salt form with a base and isolating the free base in a conventional manner.

Specifically, the base addition salts described above are formed with metals or amines, such as hydroxides of alkali metals and alkaline earth metals, or with organic amines. Examples of metals used as cations include, but are not limited to, sodium, potassium, magnesium, and calcium. Examples of suitable amines include, but are not limited to, N,N-dibenzylethylenediamine, chloroprocaine, choline, diethanolamine, ethylenediamine(ethane-1,2-diamine), N-methylglucamine and procaine. Base addition salts can be prepared by contacting the free acid form with a sufficient amount of the desired base to form the salt in a conventional manner. The free acid form can be regenerated by contacting the salt form with an acid and isolating the free acid in a conventional manner.

Specifically, the stereoisomer described above in the present invention includes enantiomeric, diastereomeric and geometric isomer forms. Some of the compounds of the present invention have cycloalkyl which may be substituted on more than one carbon atom, in which case all geometric forms thereof, including cis and trans, and mixtures thereof, are within the scope of the present invention.

Specifically, the solvate described above in the present invention refers to a physical association of the compound of the present invention with one or more solvent molecules. The physical association includes various degrees of ionic and covalent bonding, including hydrogen bonding. In some cases, the solvate can be isolated, for example, when one or more solvent molecules are incorporated into the crystal lattice of a crystalline solid. The “solvate” includes both solution phases and isolatable solvates. Representative solvates include ethanolates, methanolates, and the like. The “hydrate” is a solvate in which one or more solvent molecules are H₂O.

Specifically, the prodrug described above in the present invention refers to forms of the compound of formula I (including acetals, esters, and zwitterions) which are suitable for administration to patients without undue toxicity, irritation, allergic response and the like, and which are effective for the intended use thereof. The prodrug is converted in vivo (e.g. by hydrolysis in blood), to give the parent compound of the above formula.

Specifically, the pathogen described herein may be a microorganism, a parasite (a protozoon, a worm, etc.) or any other vector; and specifically, the above microorganism described above may be selected from one or more of a virus, a chlamydia, a rickettsia, a mycoplasma, a bacterium, a spirochete, a fungus and the like.

In one embodiment of the present invention, the pathogen described above is a virus.

Specifically, the virus described above may be a species of Herpesviridae (e.g., pseudorabies virus, bovine infectious rhinotracheitis virus, Marek’s disease virus, avian infectious laryngotracheitis virus, duck plague virus), Iridoviridae (e.g., lymphocystis disease virus), Baculoviridae (e.g., baculovirus penaei), Rhabdoviridae (e.g., rabies virus, rhabdovirus carpio), Reoviridae (e.g., avian orthoreovirus, bluetongue virus, cytoplasmic polyhedrosis virus), Birnaviridae (e.g., infectious bursal disease virus), Poxviridae (e.g., sheep pox virus, goat pox virus, myxomatosis virus), Asfarviridae (e.g., African swine fever virus), Adenoviridae (e.g., canine infectious hepatitis virus, egg drop syndrome virus), Parvoviridae (e.g., porcine parvovirus, canine parvovirus, goose parvovirus, feline panleukopenia virus, mink enteritis virus, mink Aleutian disease virus), Circoviridae (e.g., porcine circovirus), Retroviridae (e.g., avian leukosis virus, caprine arthritis/encephalomyelitis virus, equine infectious anemia virus), Orthomyxoviridae (e.g., avian influenza virus), Paramyxoviridae (e.g., Newcastle disease virus, canine distemper virus, cattle plague virus), Coronaviridae, Arteriviridae (e.g., porcine reproductive and respiratory syndrome virus), Picornaviridae (e.g., foot-and-mouth disease virus, swine vesicular disease virus, duck hepatitis virus), Caliciviridae (e.g., rabbit hemorrhagic disease virus), Flaviviridae (e.g., swine fever virus, bovine viral diarrhea virus, Japanese encephalitis virus), a prion, Filoviridae (e.g., Marburg virus, Ebola virus), Polyomaviridae, Papillomaviridae, Nimaviridae, Retroviridae, Hepadnaviridae, Papovaviridae, Bornaviridae, Bunyaviridae, Arenaviridae, Roniviridae, Hepeviridae, Astroviridae, Togaviridae, Dicistroviridae, Nodaviridae and the like.

Specifically, the virus described above may be, for example, avian infectious bronchitis virus (IBV), porcine transmissible gastroenteritis virus (TGEV), porcine epidemic diarrhea virus (PEDV), porcine hemagglutinating encephalomyelitis virus (HEV), mouse hepatitis virus (MHV), turkey bluecomb virus (TCDV), bovine coronavirus (BCV), canine coronavirus (CCV), feline infectious peritonitis virus (FIPV), rat coronavirus (RCV), rat sialodacryoadenitis coronavirus (SDAV), mink epidemic diarrhea coronavirus, sheep pox virus, goat pox virus, bovine lumpy skin disease virus, fowl pox virus, feline pox virus, infectious pustule virus, rabbit myxomatosis virus, African swine fever virus, pseudorabies virus, porcine cytomegalovirus, avian infectious laryngotracheitis virus, duck plague virus, feline viral rhinotracheitis virus, canine herpes virus, bovine infectious rhinotracheitis virus, equine rhinopneumonitis virus, Marek’s disease virus, malignant catarrhal fever virus, porcine adenovirus, canine viral hepatitis virus, feline adenovirus, porcine parvovirus, feline panleukopenia (feline distemper, feline infectious enteritis) virus, gosling plague virus, canine parvovirus, Muscovy duck parvovirus, porcine circovirus, chicken infectious anemia virus, avian leukosis virus, feline leukemia virus, bovine leukemia virus, feline immunodeficiency virus, Maedi-visna disease virus, caprine viral arthritis-encephalitis virus, equine infectious anemia virus, bovine immunodeficiency virus, feline syncytium-forming virus (foamy virus), avian viral arthritis virus, bluetongue virus, Ibaraki disease virus, Chuzan virus, African horse sickness virus, rotavirus, infectious bursal disease virus, canine viral papilloma virus, feline viral papilloma virus, Nipah disease virus, Hendra disease virus, porcine blue eye disease virus, canine parainfluenza virus, avian paramyxovirus, chicken Newcastle disease virus, canine distemper virus, peste des petits ruminants virus, cattle plague virus, avian pneumovirus, avian mumps virus (measles virus), feline paramyxovirus, rabies virus, vesicular stomatitis virus, bovine ephemeral fever/three day fever/transient fever virus, Borna virus, influenza virus, Rift Valley fever virus, Akabane virus, Hantavirus, feline enteric coronavirus, porcine hemagglutinating encephalomyelitis virus, porcine reproductive and respiratory syndrome virus, equine viral arteritis virus, foot-and-mouth disease virus, swine vesicular disease virus, porcine enterovirus, duck viral hepatitis virus, avian encephalomyelitis virus, encephalomyocarditis virus, vesicular exanthema of swine virus, feline calicivirus, rabbit viral hemorrhagic disease virus, canine hepatitis E virus, Getah virus, Japanese encephalitis B/epidemic encephalitis B virus, forest encephalitis virus, duck flavivirus, swine fever virus, bovine viral diarrhea-mucosal disease virus, border disease virus and the like.

In one embodiment of the present invention, the virus described above is a coronavirus, specifically, for example, avian infectious bronchitis virus (IBV), porcine transmissible gastroenteritis virus (TGEV), porcine epidemic diarrhea virus (PEDV), porcine hemagglutinating encephalomyelitis virus (HEV), mouse hepatitis virus (MHV), turkey bluecomb virus (TCDV), bovine coronavirus (BCV), canine coronavirus (CCV), feline infectious peritonitis virus (FIPV), rat coronavirus (RCV), rat sialodacryoadenitis coronavirus (SDAV), mink epidemic diarrhea coronavirus and the like.

In one embodiment of the present invention, the virus described above is feline infectious peritonitis virus.

As used herein, the term “animal” refers to a non-human animal, particularly to a vertebrate, and specifically to, e.g., a mammal (for example, pig, cattle, sheep, horse, donkey, dog, cat, rabbit, rodent, fox, racoon dog, mink, camel), fish, bird (for example, chicken, duck, goose, pigeon, quail, parrot, etc.), amphibian, reptile, etc., unless otherwise indicated. Particularly, the animal described above is a domestic animal, i.e., an animal raised and domesticated by humans with artificially controlled reproduction for purposes such as food, labor, fur, companionship, experiments, etc., such as an economic animal, a companion animal and a laboratory animal.

In one embodiment of the present invention, in the use described above, the animal is an economic animal, such as livestock (e.g., pig, cattle, sheep, horse, donkey, fox, racoon dog, mink, camel, etc.), poultry (chicken, duck, goose, pigeon, quail, etc.).

In one embodiment of the present invention, in the use described above, the animal is a companion animal, such as dog, cat, rabbit, rodent (e.g., guinea pig, hamster, gerbil, chinchilla, squirrel, etc.), fish, pigeon, parrot, etc.

In one embodiment of the present invention, in the use described above, the animal is a laboratory animal, such as monkey, dog, rabbit, cat, rodent, etc.

Specifically, the disease or condition described above may include avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis, mink epidemic diarrhea, sheep pox, goat pox, bovine lumpy skin disease, fowl pox, feline pox, infectious pustule, rabbit myxomatosis, African swine fever, pseudorabies, porcine cytomegalovirus infection, avian infectious laryngotracheitis, duck plague, feline viral rhinotracheitis, canine herpes virus infection, bovine infectious rhinotracheitis, equine rhinopneumonitis, Marek’s disease, malignant catarrhal fever, porcine adenovirus infection, canine viral hepatitis, feline adenovirus disease, porcine parvovirus disease, feline panleukopenia (feline distemper, feline infectious enteritis), gosling plague, canine parvovirus disease, Muscovy duck parvovirus disease, porcine circovirus disease, chicken infectious anemia, avian leukemia, feline leukemia, bovine leukemia, feline immunodeficiency disease, Maedi-visna disease, caprine viral arthritis-encephalitis, equine infectious anemia, bovine immunodeficiency virus infection, feline syncytium-forming virus (foamy virus) infection, avian viral arthritis, bluetongue, Ibaraki disease, Chuzan disease, African horse sickness, rotavirus disease, infectious bursal disease, canine viral papilloma, feline viral papilloma, Nipah disease, Hendra disease, porcine blue eye disease, canine parainfluenza virus infection, avian paramyxovirus infection, chicken Newcastle disease, canine distemper, peste des petits ruminants, cattle plague, avian pneumovirus infection, avian mumps virus infection (measles virus infection), feline paramyxovirus disease, rabies, vesicular stomatitis, bovine ephemeral fever/three day fever/transient fever, Borna disease, influenza, Rift Valley fever, Akabane disease, hantavirus disease, feline enteric coronavirus infection, porcine hemagglutinating encephalomyelitis, porcine reproductive and respiratory syndrome, equine viral arteritis, foot-and-mouth disease, swine vesicular disease, porcine enterovirus infection, duck viral hepatitis, avian encephalomyelitis, encephalomyocarditis, vesicular exanthema of swine, feline calicivirus disease, rabbit viral hemorrhagic disease, canine hepatitis E, Getah virus disease, Japanese encephalitis B/epidemic encephalitis B, forest encephalitis, duck flavivirus infection, swine fever, bovine viral diarrhea-mucosal disease, border disease and the like.

In one embodiment of the present invention, the disease or condition described above is selected from avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis and mink epidemic diarrhea.

In one embodiment of the present invention, the disease described above is feline infectious peritonitis.

Specifically, in the medicament described above, the HPK1 kinase inhibitor described above may be used as the sole active ingredient or may be used in combination with one or more additional active ingredients for the same indication or different indications, wherein the HPK1 kinase inhibitor described above and the additional active ingredients may be formulated for simultaneous, separate or sequential administration.

Specifically, the medicament described above may be in any dosage form or administration form, which can be selected by those skilled in the art according to concrete circumstances. For example, the administration form can be, but is not limited to, oral, sublingual, inhalational, subcutaneous, intramuscular, intravenous, intraperitoneal, intra-organ, intranasal, intrarectal, transdermal, ocular or rectal form; the dosage form can be, but is not limited to, tablets, pills, powders, granules, capsules, lozenges, syrups, solutions, emulsions, suspensions, controlled release formulations, aerosols, films, injections, intravenous drip infusions, transdermal absorption formulations, ointments, lotions, adhesive formulations, suppositories, nasal formulations, pulmonary formulations, eye drops and the like.

In one embodiment of the present invention, the medicament described above is an injection.

The present invention further provides use of the HPK1 kinase inhibitor described above in the preparation of a product for resisting pathogen infection in an animal.

Specifically, in the use described above, the HPK1 kinase inhibitor, the pathogen and the animal have the corresponding definitions described above in the present invention.

Specifically, the product described above may be used for either therapeutic purposes or non-therapeutic purposes.

In one embodiment of the present invention, the product described above is a pharmaceutical composition.

Specifically, in the pharmaceutical composition described above, the HPK1 kinase inhibitor described above may be used as the sole active ingredient or may be used in combination with one or more additional active ingredients for the same indication or different indications, wherein the HPK1 kinase inhibitor described above and the additional active ingredients may be formulated for simultaneous, separate or sequential administration.

Specifically, the pharmaceutical composition described above further comprises a pharmaceutically acceptable auxiliary material, in particular an auxiliary material acceptable in the field of veterinary.

Specifically, the pharmaceutical composition described above may be in any dosage form or administration form, which can be selected by those skilled in the art according to concrete circumstances. For example, the administration form can be, but is not limited to, oral, sublingual, inhalational, subcutaneous, intramuscular, intravenous, intraperitoneal, intra-organ, intranasal, intrarectal, transdermal, ocular or rectal form; the dosage form can be, but is not limited to, tablets, pills, powders, granules, capsules, lozenges, syrups, solutions, emulsions, suspensions, controlled release formulations, aerosols, films, injections, intravenous drip infusions, transdermal absorption formulations, ointments, lotions, adhesive formulations, suppositories, nasal formulations, pulmonary formulations, eye drops and the like.

In one embodiment of the present invention, the pharmaceutical composition described above is an injection.

The various forms of the pharmaceutical composition described above can be prepared according to conventional production methods in the field of veterinary drugs.

In one embodiment of the present invention, the product described above is a functional food composition.

Specifically, in the functional food composition described above, the compound of formula I may be used as the sole active ingredient, or may be used in combination with one or more additional active ingredients.

Specifically, the functional food composition described above may further comprise an animal food auxiliary material.

Specifically, the functional food composition described above may be in any form, such as tablets, pills, capsules, candies (e.g., tablet candies, gel candies, gum-based candies, etc.), solid beverages (e.g., powders, granules, etc.), liquid beverages and the like.

The various forms of the functional food composition described above can be prepared according to conventional production methods in the field of veterinary functional food.

In one embodiment of the present invention, the product described above is an additive, which can be added in a small amount or in a trace amount during the production, processing and use of the animal food (e.g., livestock and poultry feed, pet daily feed, pet snack, etc.).

The present invention further provides a method for preventing and/or treating a disease or condition caused by or associated with pathogen infection in an animal, which comprises administering to a subject an effective amount of the HPK1 kinase inhibitor described above in the present invention. Specifically, in the method described above, the HPK1 kinase inhibitor, the pathogen, the animal, and the disease or condition have the corresponding definition described above in the present invention.

The inventor of the present invention found through experiments that the HPK1 kinase inhibitor (especially the compound of formula I, such as 4-(3-{2-amino-5-[2-(1-methyl-piperidin-4-yl)thiazol-5-yl]pyridin-3-yloxymethyl}phenyl)-2-methyl-but-3-yn-2-ol) can effectively treat diseases (such as feline infectious peritonitis) caused by pathogens (specifically viruses, such as coronaviruses) infection in animals, improve animal survival, and provide enhanced commercial value and application prospects in the field of animal antiviral (specifically viruses, such as coronaviruses) medicaments.

### BRIEF DESCRIPTION OF THE DRAWINGS

FIG. 1 illustrates chest X-ray images of an affected cat in Example 1 of the present invention.

FIG. 2 illustrates the clinical survival curve in Example 2 of the present invention, wherein the day when administration started was taken as the first day.

### DETAILED DESCRIPTION

Unless otherwise defined, all scientific and technical terms used herein have the same meaning as commonly understood by those skilled in the art to which the present invention relates.

In the present invention, the term “alkyl” refers to a linear or branched hydrocarbon chain radical without unsaturated bonds, and that is attached to the rest of the molecule via a single bond. Alkyl may be substituted, for example, if alkyl is substituted with cycloalkyl, it is correspondingly “cycloalkylalkyl”, such as cyclopropylmethyl; if alkyl is substituted with halogen, it is correspondingly “haloalkyl”; if alkyl is substituted with aryl, it is correspondingly “aralkyl”, such as benzyl, benzhydryl or phenethyl; if alkyl is substituted with heterocyclyl, then it is correspondingly “heterocyclylalkyl”; and so on. The C₀₋₁₀ alkyl in the present invention refers to alkyl containing 0 to 10 (e.g., 0, 1, 2, 3, 4, 5, 6, 7, 8, 9 or 10) carbon atoms, such as methyl, ethyl, n-propyl, isopropyl, n-butyl, isobutyl, tert-butyl, n-pentyl, isopentyl, neopentyl, tert-pentyl, n-hexyl, isohexyl, etc., wherein C₀ alkyl refers to H.

The term “cycloalkyl” refers to an alicyclic hydrocarbon, for example, containing 1 to 4 monocyclic and/or fused rings and 3 to 18 carbon atoms, preferably 3 to 10 carbon atoms (such as cyclopropyl, cyclohexyl or adamantyl), and the C₃₋₁₀ cycloalkyl in the present invention refers to cycloalkyl containing 3 to 10 (e.g., 3, 4, 5, 6, 7, 8, 9 or 10) carbon atoms, such as cyclopropyl, cyclobutyl, cyclopentyl, cyclohexyl, etc.

The term “aryl” refers to a monocyclic or polycyclic radical, including polycyclic radicals containing monoaryl and/or fused aryl groups, for example, containing 1 to 3 monocyclic or fused rings and 6 to 18 (e.g., 6, 8, 10, 12, 14, 16 or 18) carbon ring atoms, such as phenyl, naphthyl, biphenyl, indenyl, etc.

The term “heterocyclyl” includes heteroaromatic and heteroalicyclic groups containing 1 to 3 monocyclic and/or fused rings and 3 to 18 (e.g., 3, 4, 5, 6, 7, 8, 9, 10, 11, 12, 13, 14, 15, 16, 17 or 18) ring atoms. Preferred heteroaromatic groups and heteroalicyclic groups contain 5 to about 10 ring atoms. Suitable heteroaryl in the compound of the present invention contains 1, 2 or 3 heteroatoms which may be selected from N, O and S atoms.

The groups described above in the present invention may be substituted with one or more suitable groups at one or more available positions, such as, but not limited to, OR’, =O, SR’, SOR’, SO₂R′, OSO₂R′, OSO₃R′, NO₂, NHR’, N(R′)₂, =N-R′, N(R′)COR’, N(COR′)₂, N(R′)SO₂R′, N(R′)C(=NR′)N(R′)R′, N₃, CN, halogen, COR’, COOR’, OCOR’, OCOOR’, OCONHR’, OCON(R′)₂, CONHR’, CON(R′)₂, CON(R′)OR’, CON(R′)SO₂R′, PO(OR’)₂, PO(OR’)R′, PO(OR’)(N(R′)R′), C₁-C₁₂ alkyl, C₃-C₁₀ cycloalkyl, C₂-C₁₂ alkenyl, C₂-C₁₂ alkynyl, aryl and heterocyclyl. Each R′ group is independently selected from: hydrogen, OH, NO₂, NH₂, SH, CN, halogen, COH, CO alkyl, COOH, C₁-C₁₂ alkyl, C₃-C₁₀ cycloalkyl, C₂-C₁₂ alkenyl, C₂-C₁₂ alkynyl, aryl, and heterocyclyl; wherein these groups themselves may be substituted, and the substituents may be selected from the foregoing list. The term “halogen”, “halogenated” or “halo” refers to bromo, chloro, iodo or fluoro.

The term “treating” includes eradicating, removing, reversing, alleviating, altering or controlling a disease or condition after its onset.

The term “preventing” refers to the ability to avoid, minimize, or make difficult the onset or progression of a disease or condition prior to its onset through treatment.

The term “pathogen” refers to a microorganism (including virus, chlamydia, rickettsia, mycoplasma, bacterium, spirochete, fungus, and the like), parasite (protozoon, worm, and the like) or other vectors that can cause infections or diseases in humans or animals and plants. Among these, the bacterium may be, for example, a Gram-positive cocci, an enterobacterium, a vibrio, a pasteurella, a Gram-negative aerobic bacterium, a Gram-negative microaerophilic and anaerobic bacterium, a Gram-positive non-spore-forming bacillus, a Gram-positive spore-forming bacillus and mycobacterium; the fungus can be, for example, a candida, a cryptococcus and an aspergillus. In the present invention, the pathogen generally refers to a microorganism, a parasite or any other vector that can cause infection or diseases in animals. In one embodiment of the present invention, the pathogen is a virus. The term “viral infection” refers to the process by which a virus invades the body through a variety of pathways and proliferates in susceptible host cells. The infected body may demonstrate different clinical types. The infection can be classified into apparent infection and latent infection according to the presence or absence of symptoms. An infection where the virus proliferates in host cells but no remarkable clinical symptoms are observed due to the small amount or weak toxicity of the invading virus or the strong resistance of the body is referred to as latent infection. Although a latent infection does not demonstrate clinical symptoms, the virus still proliferates in vivo and spreads new viruses to others and becomes an important source of infection. Therefore, resisting viral infection is also required for a host with latent infection. An infection where the virus proliferates in host cells and causes remarkable clinical symptoms due to the large amount or strong toxicity of the invading virus or the weak resistance of the body is referred to as latent infection.

As used herein, the term “animal” refers to a non-human animal, particularly to a vertebrate, and specifically to, e.g., a mammal (for example, pig, cattle, sheep, horse, donkey, dog, cat, rabbit, rodent, fox, racoon dog, mink, camel), fish, bird (for example, chicken, duck, goose, pigeon, quail, parrot, etc.), amphibian and reptile, unless otherwise indicated. Particularly, the animal described above is a domestic animal, i.e., an animal raised and domesticated by humans with artificially controlled reproduction for purposes such as food, labor, fur, companionship and experiment, such as an economic animal, a companion animal and a laboratory animal. The term “economic animal” refers to an animal, such as livestock, poultry and the like, that is raised for meat, milk, fur, labor or other economic purposes; the term “livestock” refers to a domestic animal that is raised and utilized by humans for breeding, and is beneficial to agricultural production; the term “poultry” refers to an avian animal that is fed by humans mainly for meat, eggs, feather or other purposes; the term “companion animal” refers to an animal raised for mental purposes (e.g., for appreciation and companion purposes) rather than economic purposes; the term “laboratory animal” refers to an animal raised for scientific application purposes.

The technical schemes of the present invention will be clearly and completely described below with reference to the examples of the present invention, and it is obvious that the described examples are only a part of the examples of the present invention but not all of them. Based on the examples of the present invention, all other examples obtained by those of ordinary skill in the art without creative work shall fall within the protection scope of the present invention.

### Example 1

### 1. Procedures

Lele, a British shorthair cat, 8 months old, female, unneutered, weighing 2.5 kg. The cat had symptoms of elevated body temperature, mental depression, anorexia, and enlarged abdominal circumference in January, 2020. One week later, the cat was admitted. On January 18, the cat was diagnosed with feline infectious peritonitis (FIP) positive. After being treated with GS-441524 for 3 days, the cat was then given furosemide, Synulox®, prednisolone and itraconazole (P.O., q24h, 10 mg/kg) for 1 month. The drugs were discontinued after ascites regressed. Early in April, the disease recurred, the cat showed decreased food intake and listlessness. X-ray examination suggested hydrothorax, and PCR test showed feline coronavirus positive (the CT value was 25.11). The cat was enrolled on April 8, and was treated with the drug of the present invention at 2.0 mg/kg (SC, q24h) on days 1-14 (D1-14). Proper supportive treatments were given at the same time, including adjuvant treatment such as Moringa (a hepatoprotective drug), Lysinphirst (lysine) and Doctor Dolittle® (lactoferrin).

### 2. Preparation and Use

The pharmaceutically active compound described above is a pure powder have the following structure. The compound was added to a 0.9% normal saline (50 mL of endotoxin-free water and 0.45 g of NaCl), and the resulting mixture was uniformly mixed and filtered through a filter membrane to give a clarified solution. The diluted compound was stored in a plastic-sealed sterile vial, stored in a refrigerator at 4° C., gently brought to room temperature before injection, and administered using a disposable syringe. The injection ranged across the back, starting from 2 cm posterior to the scapula to the middle of the lumbar spine, half of the distance of the adjacent chest to flank.

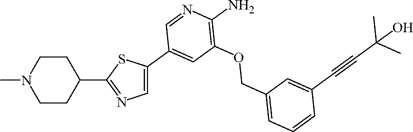 [SMILES: CN1CCC(c2ncc(-c3cnc(N)c(OCc4cccc(C#CC(C)(C)O)c4)c3)s2)CC1]

### 3. Results

The treatment was given by injection in the morning every day, body temperature, appetite, mental state and respiratory state were measured and evaluated at 15:00 pm, and urination and defecation conditions were recorded. The results are shown in Table 1.

TABLE 1

Experimental results

| No. | Time | Body temperature Morning | Body temperature Afternoon | Mental state | Food intake | Respiratory state | Defecation condition | Notes |
|---|---|---|---|---|---|---|---|---|
| D0 | 4-8 | | 38.5 | Listless - 3 | Decreased intake - 3 | Normal - 5 | Normal - 5 | 2.5 kg body weight at enrollment |
| D1 | 4-9 | | 38.6 | Slightly improved - 3.5 | Decreased intake - 3 | Normal | Normal | |
| D2 | 4-10 | | 38.1 | Improved - 4 | Improved - 4 | Normal | Normal | |
| D3 | 4-11 | | 38 | Good - 5 | Good - 5 | Normal | Normal | |
| D4 | 4-12 | | 38.3 | Good | Good | Normal | Normal | |
| D5 | 4-13 | | 38.3 | Good | Good | Normal | Normal | |
| D6 | 4-14 | | 38.3 | Good | Good | Normal | Normal | |
| D7 | 4-15 | | 38.1 | Good | Good | Normal | Normal | 2.75 kg body weight |
| D8 | 4-16 | | 38 | Good | Good | Normal | Normal | |
| D9 | 4-17 | | 38.2 | Good | Good | Normal | Normal | |
| D10 | 4-18 | | 38.3 | Good | Good | Normal | Normal | |
| D11 | 4-19 | | 38.4 | Good | Good | Normal | Normal | |
| D12 | 4-20 | | 38.5 | Good | Good | Normal | Normal | |
| D13 | 4-21 | 38.9 | 38.5 | Good | Good | Normal | Normal | |
| D14 | 4-22 | 38.4 | 38.4 | Good | Good | Normal | Normal | 2.75 kg body weight |

The following procedures were performed on D0, D7 and D14 after enrollment:

1 mL of serum sample was collected and frozen for multi-factor assay. The results are shown in Table 2.

TABLE 2

Blood routine test results

| Blood routine test | | Normal range | D0 | D7 | D14 |
|---|---|---|---|---|---|
| WBC | White blood cell | 5.5~19.5 | 13.5 | 18.7 | 19.7 |
| RBC | Red blood cell | 5.0~10.0 | 10.5 | 7.94 | 7.94 |
| HGB | Hemoglobin | 80~150 | 117 | 94 | 91 |
| HCT | Hematocrit | 24.0~45.0 | 37.5 | 28.9 | 29.9 |
| MCV | Mean corpuscular volume | 39.0~55.0 | 35.9 | 36.4 | 37.7 |
| MCH | Mean corpuscular hemoglobin | 12.5~17.5 | 11.2 | 11.8 | 11.5 |

### 3) Chest X-ray

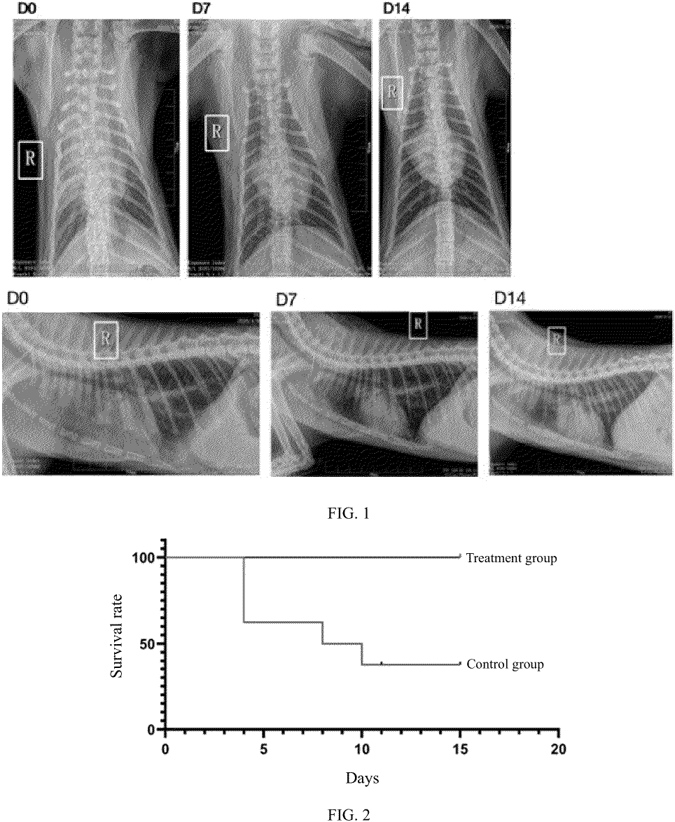

As shown in FIG. 1.

### 4) Analysis of Results

The above results suggested wet FIP (in the chest), showing hydrothorax, listlessness and anorexia, which were typical clinical symptoms of feline infectious peritonitis.

Because the cat had no fever symptom at enrollment, the fever alleviation effect cannot be observed during treatment, and the body temperature was always maintained at in the normal range, 38-38.5° C., throughout the treatment. After 2 weeks of treatment, the cat demonstrated remarkable clinical response with improved appetite and mental state, and weight gain. Since about 7 days after the treatment, the hydrothorax completely regressed. The body weight increased by 9% during and after the treatment.

### Example 2

A total of 15 cats with clinically confirmed FIP were enrolled, with hydrothorax/ascites, positive CT value by PCR, and no neurological symptoms. 8 cats were allocated into the control group, and 7 cats were allocated into the treatment group.

The groups were pre-treated with GS-441524 for 3 days. For the control group, necessary supportive treatment was given on D1-D14. For the treatment group, after 3 days of observation, necessary supportive treatment and the drug of the present invention (2.0 mg/kg, SC, q24 h) were given on D1-D14.

The occurrence of death or neurological symptoms was judged as clinical death, and the clinical survival curve was plotted after 14 days. The results are shown in FIG. 2. Log Rank (Mantel-Cox) and Breslow (Generalized Wilcoxon) methods were used for validity test, and both methods indicated a significant difference (P < 0.05).

The above description is only for the purpose of illustrating the preferred example of the present invention, and is not intended to limit the scope of the present invention. Any modifications, equivalents and the like made without departing from the spirit and principle of the present invention should be included in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1. Use of an HPK1 kinase inhibitor in the preparation of a medicament for preventing and/or treating a disease or condition caused by or associated with a pathogen infection in an animal.

2. The use according to claim 1, wherein the HPK1 kinase inhibitor is a small molecule inhibitor;
preferably, the small molecule HPK1 kinase inhibitor is a compound of the following general formula I, or a pharmaceutically acceptable salt, a stereoisomer, an ester, a prodrug, a solvate or a deuterated compound thereof: 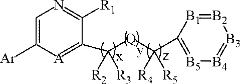
wherein,
A is selected from C and N;
Ar is selected from aromatic five-membered heterocyclic group, aromatic six-membered heterocyclic group and phenyl;
R₁ is selected from: —H, halogen, —NO₂, —CN, C₁₋₅ linear/branched alkyl, C₃₋₁₀ cycloalkyl, -N(C₀₋₁₀ alkyl)(C₀₋₁₀ alkyl), —CF₃, —OCF₃, —OCHF₂, —OCH₂F and -OC₀₋₁₀ alkyl;
R₂ and R₃ are independently selected from: —H, halogen and C₁₋₁₀ linear/branched alkyl;
R₄ and R₅ are independently selected from: —H, deuterium, halogen, C₁₋₁₀ linear/branched alkyl and deuterated C₁₋₁₀ linear/branched alkyl;
Q is selected from O and S;
x and z are independently selected from integers between 0 and 6;
y is 0 or 1;
B₁, B₂, B₃, B₄ and B₅ are independently selected from CR₆ and N; wherein each R₆ is independently selected from: —H, halogen, —CN, -OC₀₋₁₀ alkyl, C₁₋₁₀ linear/branched alkyl, heteroalkyl containing O or N, -N(C₀₋₁₀ alkyl)(C₀₋₁₀ alkyl), C₃₋₁₀ cycloalkyl, —C≡C—R₇, —O heterocycloalkyl, -N heterocycloalkyl, -COO(C₀₋₁₀ alkyl)(C₀₋₁₀ alkyl), -CO(C₀₋₁₀ alkyl)(C₀₋₁₀ alkyl), -CON(C₀₋₁₀ alkyl)(C₀₋₁₀ alkyl) and -N(C₀₋₁₀ alkyl)CO(C₀₋₁₀ alkyl)(C₀₋₁₀ alkyl), or two R₆, together with carbon atoms therebetween, form C₃₋₈ cycloalkyl or C₃₋₈ heterocycloalkyl containing —O— or —S—, -N heterocycloaryl, —O heterocycloaryl, —S heterocycloaryl or phenyl;
R₇ is selected from H, C₁₋₅ linear/branched alkyl, C₃₋₁₀ cycloalkyl and 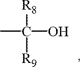
wherein R₈ and ₉ are independently selected from: —H, —CF₃, —CHF₂H, —CH₂F, C₁₋₁₀ linear/branched alkyl, -CH=C(C₀₋₁₀ alkyl)(C₀₋₁₀ alkyl), -C≡C(C₀₋₁₀ alkyl), C₃₋₁₀ cycloalkyl, -CO(C₀₋₁₀ alkyl)(C₀₋₁₀ alkyl), -CO(C₀₋₁₀ alkyl)(C₀₋₁₀ alkyl), -CON(C₀₋₁₀ alkyl)(C₀₋₁₀ alkyl), -N(C₀₋₁₀ alkyl)CO(C₀₋₁₀ alkyl)(C₀₋₁₀ alkyl), aromatic five-membered cyclic group and aromatic six-membered cyclic group, or R₈ and R₉, together with carbon atoms between R₈ and R₉, form C₃₋₈ cycloalkyl or C₃₋₈ heterocycloalkyl containing —O— or —S—, C₄₋₉ fused cycloalkyl, C₅₋₁₀ spiro cycloalkyl, C₄₋₉ bridged cycloalkyl, C₃₋₇ cyclolactam, C₃₋₇ cyclic lactone or C₃₋₇ cyclic ketone.

3. The use according to claim 2, wherein the aromatic five-membered heterocyclic group is selected from: furyl, thienyl, pyrrolyl, pyrazolyl, imidazolyl, oxazolyl, thiazolyl and selenothiazolyl; and/or the aromatic six-membered heterocyclic group is selected from: pyridyl, pyridazinyl, pyrimidinyl and pyrazinyl.

4. The use according to claim 2, wherein Ar is selected from: 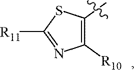 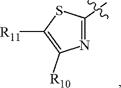 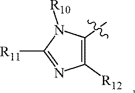 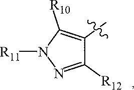 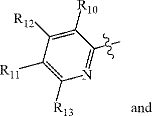 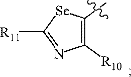
wherein R₁₀, R₁₂ and R₁₃ are independently selected from: —H, C₁₋₁₀ linear/branched alkyl, -N(C₀₋₁₀ alkyl)(C₀₋₁₀ alkyl), -OC₀₋₁₀ alkyl and C₃₋₁₀ cycloalkyl;
R₁₁ is selected from: —H, —O heterocycloalkyl, —N heterocycloalkyl, C₁₋₁₀ linear/branched alkyl, C₃₋₁₀ cycloalkyl, -OC₀₋₁₀ alkyl, -N(C₀₋₁₀ alkyl)(C₀₋₁₀ alkyl), -SO₂(C₀₋₁₀ alkyl), -O(C₀₋₁₀ alkyl), —O—phenyl, -S(C₀₋₁₀ alkyl), —N heterocycloaryl, —O heterocycloaryl and —S heterocycloaryl.

5. The use according to claim 4, wherein R₁₁ is selected from:

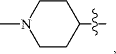

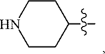

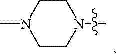

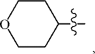

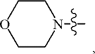

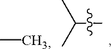

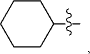

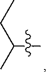

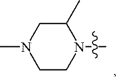

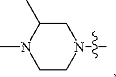

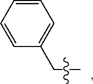

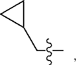

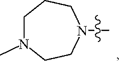

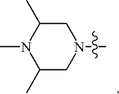

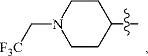

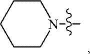

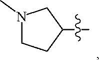

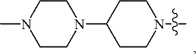

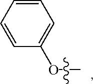

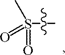

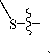

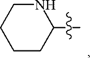

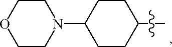

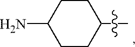

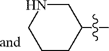

6. The use according to claim 2, wherein at least one of B₁, B₂, B₃, B₄ and ₅ is N; or
B₁, B₂, B₃, B₄ and B₅ are all CR₆, and in the general formula I, 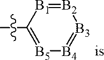 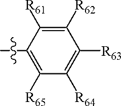
wherein R₆₁, R₆₂, R₆₃, R₆₄ and R₆₅ are independently selected from: —H, halogen, —CN, -OC₀₋₁₀ alkyl, C₁₋₁₀ linear/branched alkyl, heteroalkyl containing O or N, -N(C₀₋₁₀ alkyl)(C₀₋₁₀ alkyl), C₃₋₁₀ cycloalkyl, —C≡C—R₇, —O heterocycloalkyl and —N heterocycloalkyl, or R₆₁ and R₆₂, R₆₂ and R₆₃, R₆₃ and R₆₄, R₆₄ and R₆₅, together with carbon atoms therebetween, form C₃₋₈ cycloalkyl or C₃₋₈ heterocycloalkyl containing —O— or —S—, —N heterocycloaryl, —O heterocycloaryl, —S heterocycloaryl or phenyl.

7. The use according to claim 6, wherein R₆₃ is selected from: —H, halogen, -OC₀₋₁₀ alkyl, C₁₋₁₀ linear/branched alkyl, -N(C₀₋₁₀ alkyl)(C₀₋₁₀ alkyl) and C₃₋₁₀ cycloalkyl; preferably, R₆₃ is selected from: —H, halogen, -OC₀₋₁₀ alkyl and C₁₋₁₀ linear/branched alkyl; and more preferably, R₆₃ is selected from: —H, —F and —OCH₃.

8. The use according to claim 6, wherein R₆₂ is selected from: —H, halogen, -OC₀₋₁₀ alkyl, —CN, C₃₋₁₀ cycloalkyl and -C≡C-R₇;
preferably, R₆₂ is selected from: —H, —F, —Cl, —OCH₃, —CN, 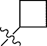
and -C≡C-R₇; and more preferably, R₆₂ is C≡C-R₇.

9. The use according to claim 6, wherein R₆₁, R₆₄ and R₆₅ are independently selected from: —H, halogen, —CN, -OC₀₋₁₀ alkyl, C₁₋₁₀ linear/branched alkyl, -N(C₀₋₁₀ alkyl)(C₀₋₁₀ alkyl), C₃₋₁₀ cycloalkyl, -C≡C-R₇, —O heterocycloalkyl, —N heterocycloalkyl and C₁₋₅ linear/branched alkyl containing O and N, or R₆₄ and R₆₅, together with carbon atoms attach to R₆₄ and R₆₅, form C₃₋₈ cycloalkyl or C₃₋₈ heterocycloalkyl containing —O— or —S—;
preferably, R₆₁, R₆₄ and R₆₅ are independently selected from: —H, halogen, C₁₋₃ linear/branched alkyl, -OC₀₋₃ alkyl and C₁₋₃ linear/branched alkyl containing N, or R₆₄ and R₆₅, together with carbon atoms attach to R₆₄ and R₆₅, form C₃₋₈ cycloalkyl or C₃₋₈ heterocycloalkyl containing —O—; and
more preferably, R₆₁, R₆₄ and R₆₅ are independently selected from: —H, —F, —Cl, —CH₃, —CH2NH2, —CN and —OCH₃, or R₆₄ and R₆₅, together with carbon atoms attach to R₆₄ and R₆₅, form five-membered cycloalkyl containing —O—.

10. The use according to claim 8, wherein R₈ and R₉ are independently selected from:
—H, —CF₃, —CHF₂, —CH₂F, —CH₃, —CH₂CH₃, —CH═CH₂, 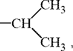 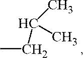 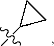 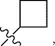 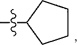 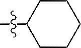 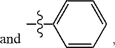
or R₈ and R₉, together with carbon atoms between R₈ and R₉, form 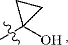 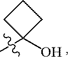 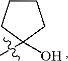 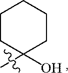 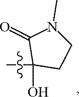 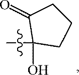 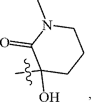 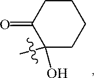 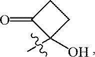 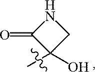 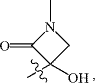 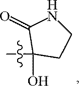 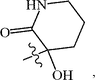 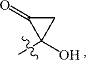 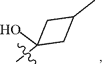 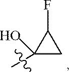 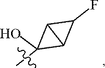 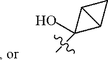
.

11. The use according to claim 2, wherein the compound is selected from: 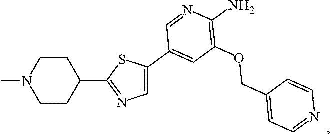 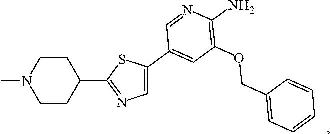 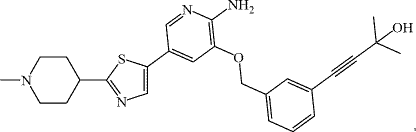 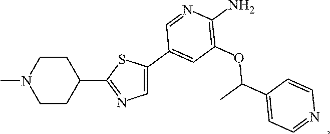 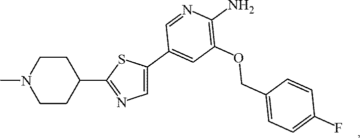 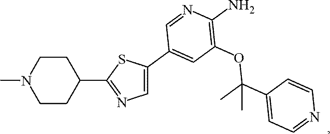 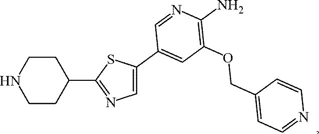 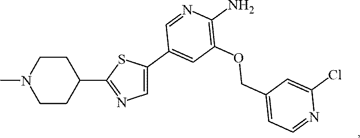 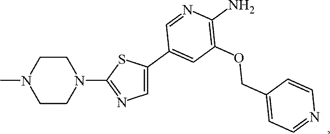 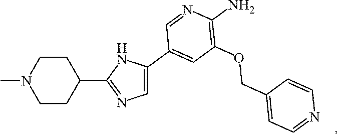 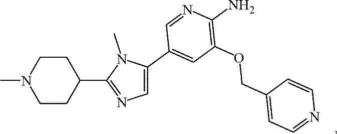 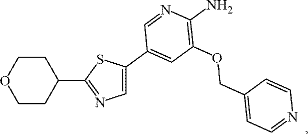 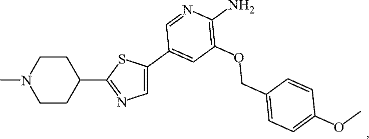 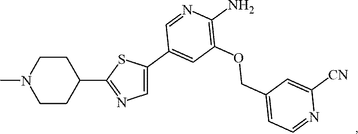 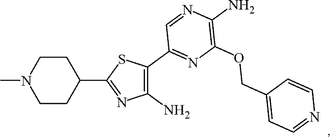 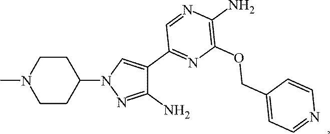 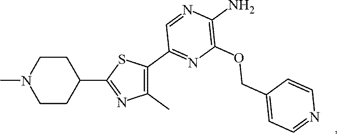 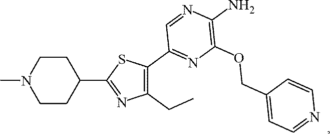 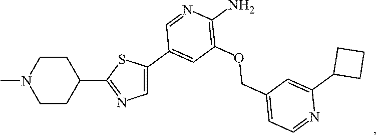 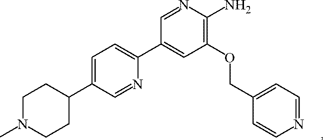 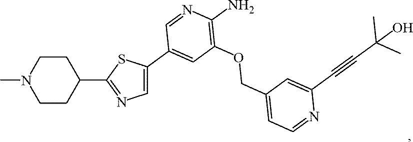 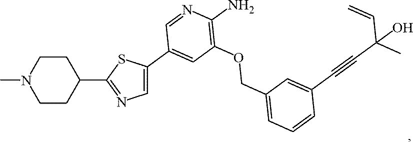 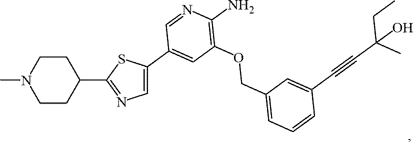 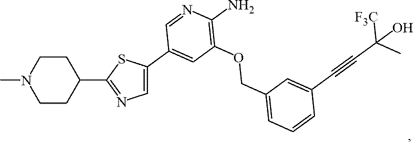 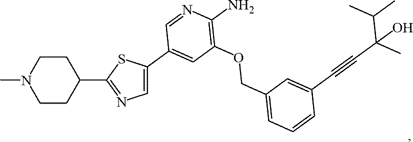 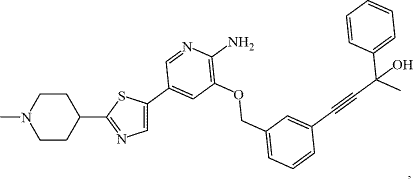 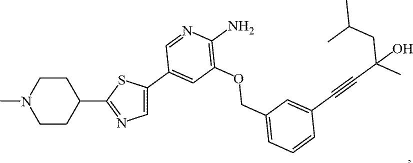 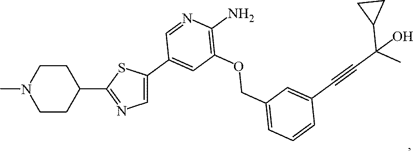 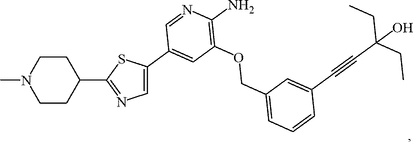 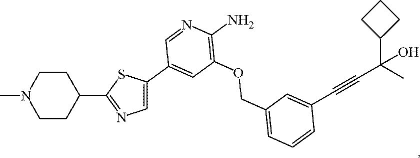 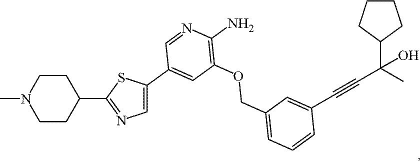 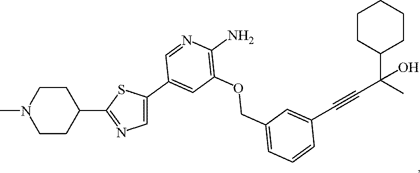 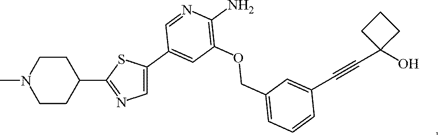 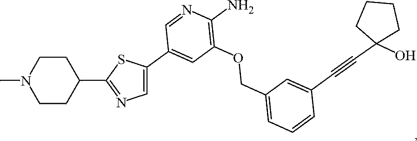 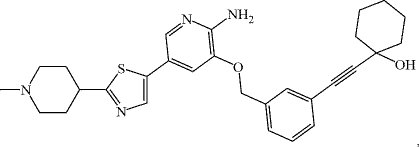 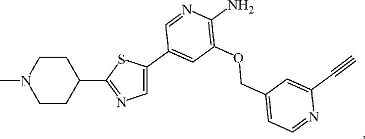 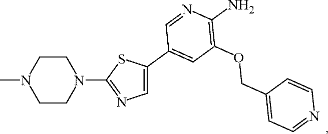 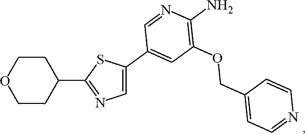 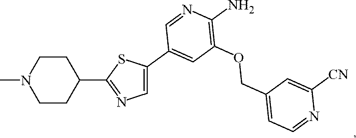 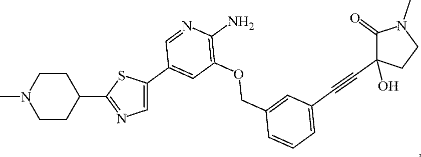 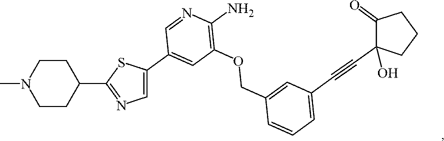 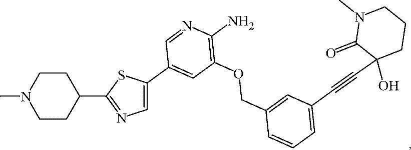 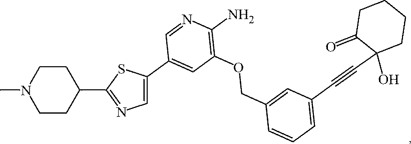 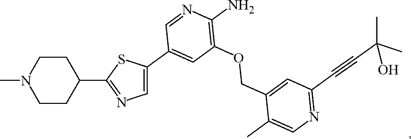 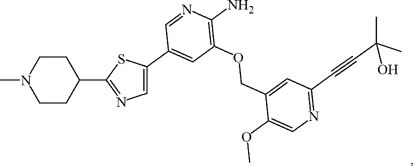 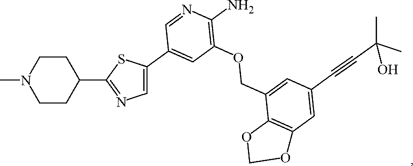 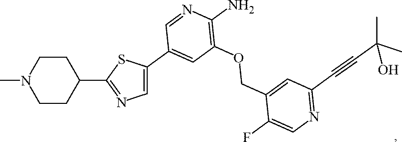 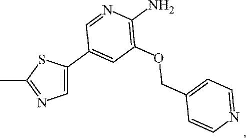 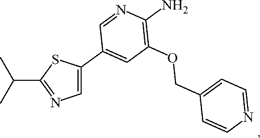 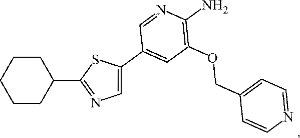 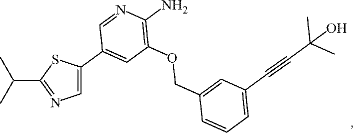 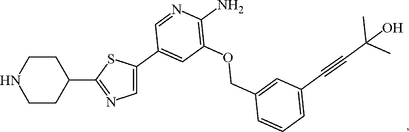 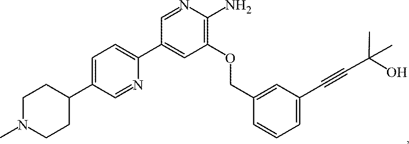 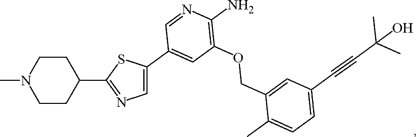 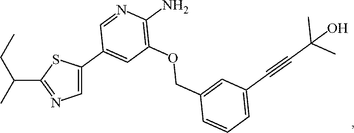 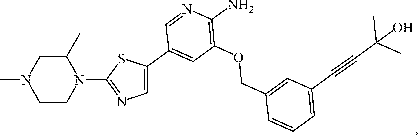 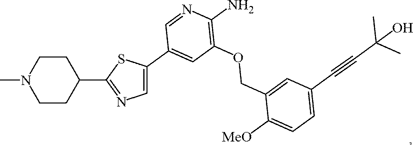 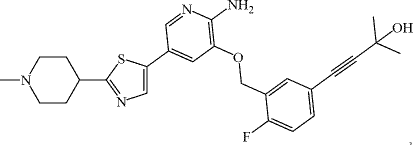 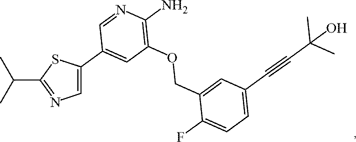 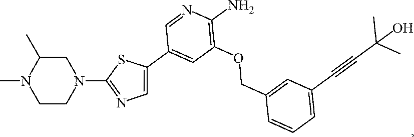 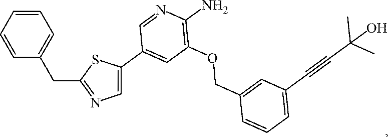 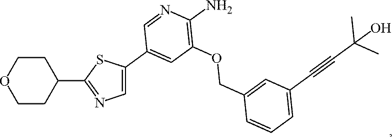 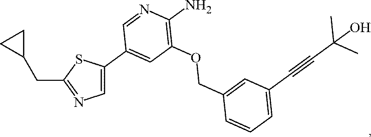 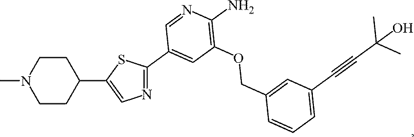 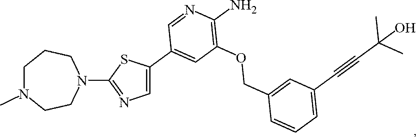 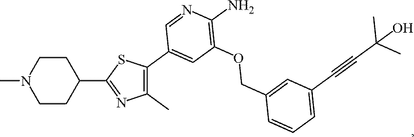 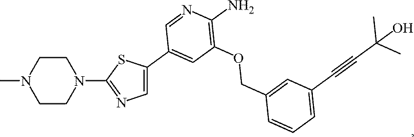 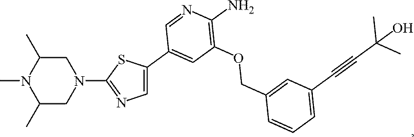 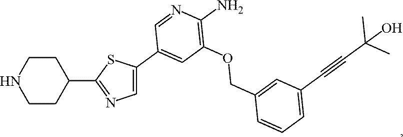 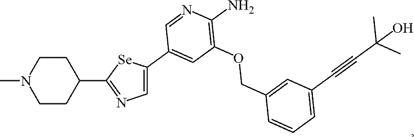 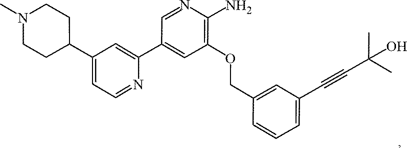 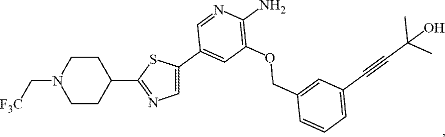 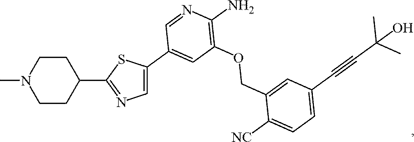 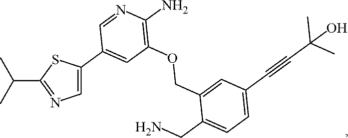 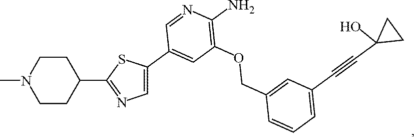 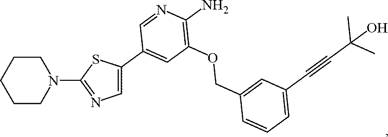 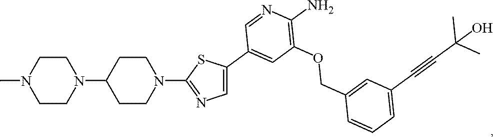 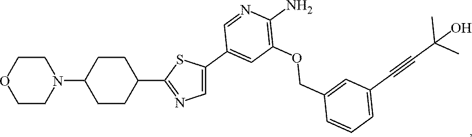 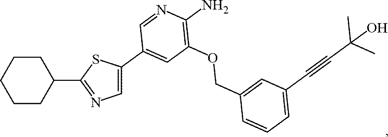 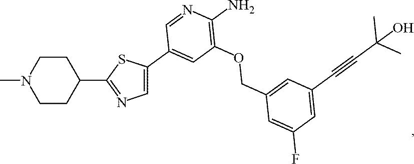 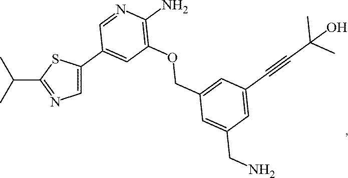 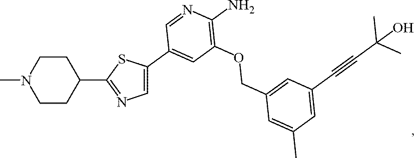 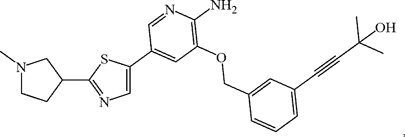 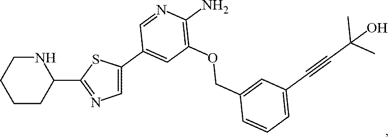 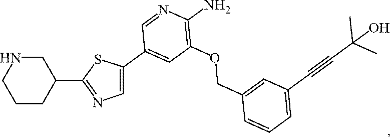 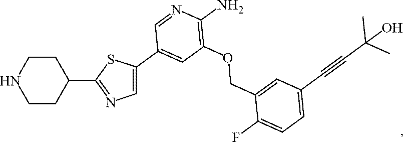 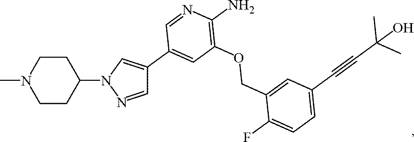 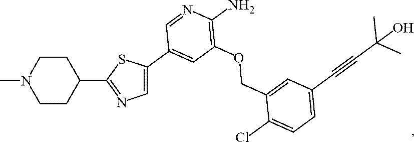 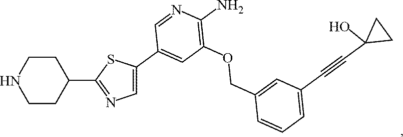 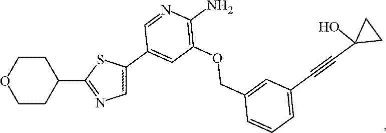 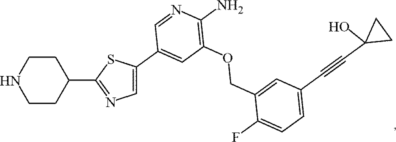 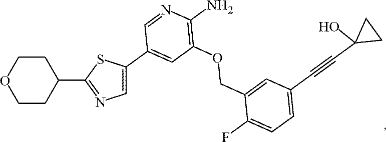 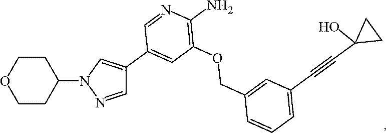 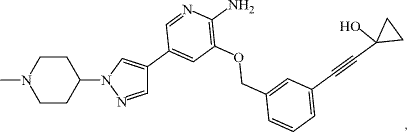 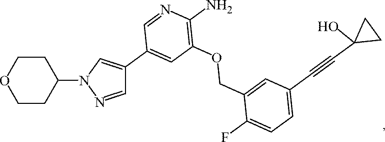 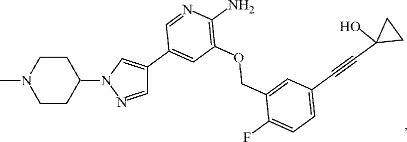 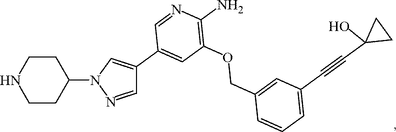 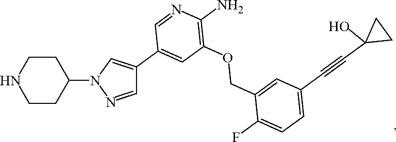 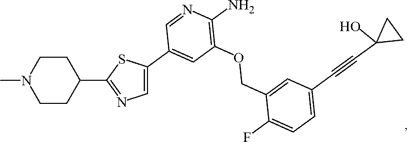 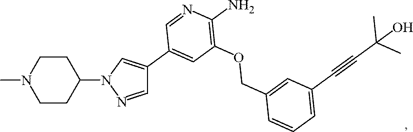 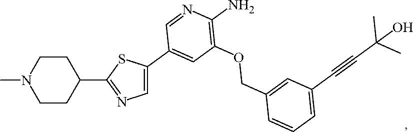 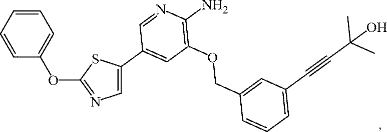 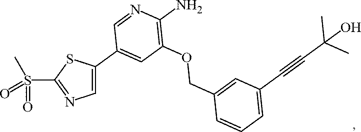 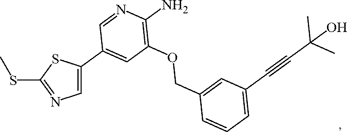 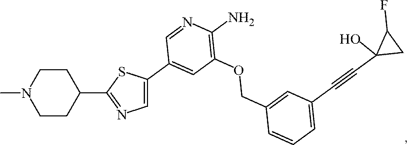 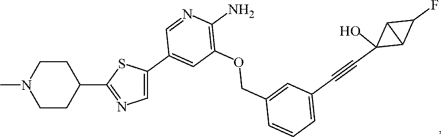 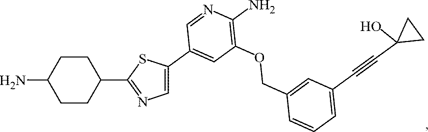 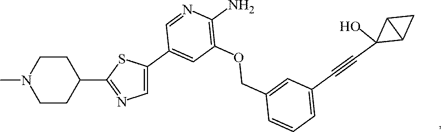 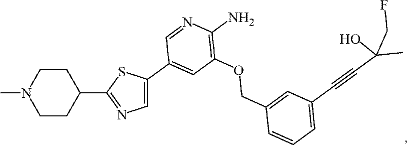 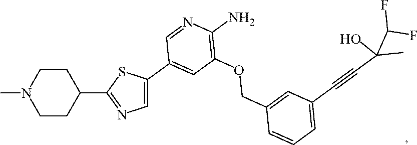 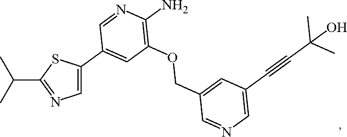 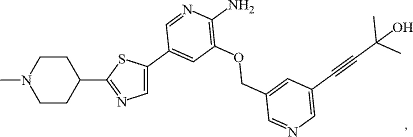 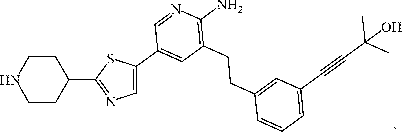 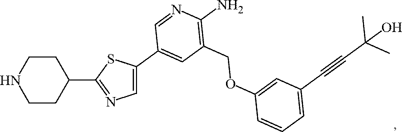 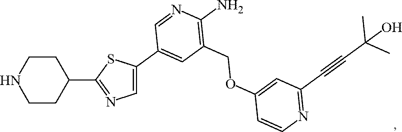 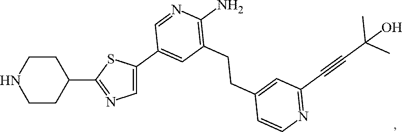 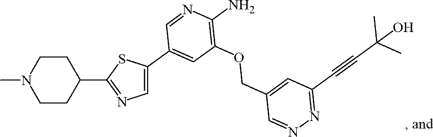 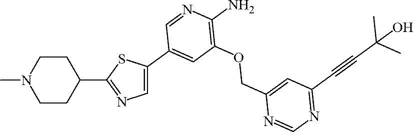 [SMILES: CN1CCC(c2ncc(-c3cnc(N)c(OCc4cc(C#CC(C)(C)O)ncn4)c3)s2)CC1]
.

12. The use according to claim 1, wherein the pathogen is a virus;
preferably, the virus is a species selected from Herpesviridae, Iridoviridae, Baculoviridae, Rhabdoviridae, Reoviridae, Birnaviridae, Poxviridae, Asfarviridae, Adenoviridae, Parvoviridae, Circoviridae, Retroviridae, Orthomyxoviridae, Paramyxoviridae, Coronaviridae, Arteriviridae, Picomaviridae, Caliciviridae, Flaviviridae, a prion, Filoviridae, Polyomaviridae, Papillomaviridae, Nimaviridae, Retroviridae, Hepadnaviridae, Papovaviridae, Bornaviridae, Bunyaviridae, Arenaviridae, Roniviridae, Hepeviridae, Astroviridae, Togaviridae, Dicistroviridae and Nodaviridae; and
preferably, the virus is a coronavirus.

13. The use according to claim 12, wherein the virus is selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus, mink epidemic diarrhea coronavirus, sheep pox virus, goat pox virus, bovine lumpy skin disease virus, fowl pox virus, feline pox virus, infectious pustule virus, rabbit myxomatosis virus, African swine fever virus, pseudorabies virus, porcine cytomegalovirus, avian infectious laryngotracheitis virus, duck plague virus, feline viral rhinotracheitis virus, canine herpes virus, bovine infectious rhinotracheitis virus, equine rhinopneumonitis virus, Marek’s disease virus, malignant catarrhal fever virus, porcine adenovirus, canine viral hepatitis virus, feline adenovirus, porcine parvovirus, feline panleukopenia virus, gosling plague virus, canine parvovirus, Muscovy duck parvovirus, porcine circovirus, chicken infectious anemia virus, avian leukosis virus, feline leukemia virus, bovine leukemia virus, feline immunodeficiency virus, Maedi-visna disease virus, caprine viral arthritis-encephalitis virus, equine infectious anemia virus, bovine immunodeficiency virus, feline syncytium-forming virus, avian viral arthritis virus, bluetongue virus, Ibaraki disease virus, Chuzan virus, African horse sickness virus, rotavirus, infectious bursal disease virus, canine viral papilloma virus, feline viral papilloma virus, Nipah disease virus, Hendra disease virus, porcine blue eye disease virus, canine parainfluenza virus, avian paramyxovirus, chicken Newcastle disease virus, canine distemper virus, peste des petits ruminants virus, cattle plague virus, avian pneumovirus, avian mumps virus, feline paramyxovirus, rabies virus, vesicular stomatitis virus, bovine ephemeral fever/three day fever/transient fever virus, Boma virus, influenza virus, Rift Valley fever virus, Akabane virus, Hantavirus, feline enteric coronavirus, porcine hemagglutinating encephalomyelitis virus, porcine reproductive and respiratory syndrome virus, equine viral arteritis virus, foot-and-mouth disease virus, swine vesicular disease virus, porcine enterovirus, duck viral hepatitis virus, avian encephalomyelitis virus, encephalomyocarditis virus, vesicular exanthema of swine virus, feline calicivirus, rabbit viral hemorrhagic disease virus, canine hepatitis E virus, Getah virus, Japanese encephalitis B/epidemic encephalitis B virus, forest encephalitis virus, duck flavivirus, swine fever virus, bovine viral diarrhea-mucosal disease virus and border disease virus;
preferably, the virus is a coronavirus selected from avian infectious bronchitis virus, porcine transmissible gastroenteritis virus, porcine epidemic diarrhea virus, porcine hemagglutinating encephalomyelitis virus, mouse hepatitis virus, turkey bluecomb virus, bovine coronavirus, canine coronavirus, feline infectious peritonitis virus, rat coronavirus, rat sialodacryoadenitis coronavirus and mink epidemic diarrhea coronavirus; and
more preferably, the virus is feline infectious peritonitis virus.

14. The use according to claim 12, wherein the animal is a domestic animal; and preferably, the animal is an economic animal, a companion animal or a laboratory animal.

15. The use according to claim 12, wherein the disease or condition include avian infectious bronchitis, porcine transmissible gastroenteritis, porcine epidemic diarrhea, canine coronavirus disease, porcine hemagglutinating encephalomyelitis, hepatitis, encephalitis and enteritis caused by mouse hepatitis virus, turkey bluecomb, neonatal calf diarrhea, bovine blood dysentery, feline infectious peritonitis, rat sialodacryoadenitis, mink epidemic diarrhea, sheep pox, goat pox, bovine lumpy skin disease, fowl pox, feline pox, infectious pustule, rabbit myxomatosis, African swine fever, pseudorabies, porcine cytomegalovirus infection, avian infectious laryngotracheitis, duck plague, feline viral rhinotracheitis, canine herpes virus infection, bovine infectious rhinotracheitis, equine rhinopneumonitis, Marek’s disease, malignant catarrhal fever, porcine adenovirus infection, canine viral hepatitis, feline adenovirus disease, porcine parvovirus disease, feline panleukopenia (feline distemper, feline infectious enteritis), gosling plague, canine parvovirus disease, Muscovy duck parvovirus disease, porcine circovirus disease, chicken infectious anemia, avian leukemia, feline leukemia, bovine leukemia, feline immunodeficiency disease, Maedi-visna disease, caprine viral arthritis-encephalitis, equine infectious anemia, bovine immunodeficiency virus infection, feline syncytium-forming virus (foamy virus) infection, avian viral arthritis, bluetongue, Ibaraki disease, Chuzan disease, African horse sickness, rotavirus disease, infectious bursal disease, canine viral papilloma, feline viral papilloma, Nipah disease, Hendra disease, porcine blue eye disease, canine parainfluenza virus infection, avian paramyxovirus infection, chicken Newcastle disease, canine distemper, peste des petits ruminants, cattle plague, avian pneumovirus infection, avian mumps virus infection, feline paramyxovirus disease, rabies, vesicular stomatitis, bovine ephemeral fever/three day fever/transient fever, Boma disease, influenza, Rift Valley fever, Akabane disease, hantavirus disease, feline enteric coronavirus infection, porcine hemagglutinating encephalomyelitis, porcine reproductive and respiratory syndrome, equine viral arteritis, foot-and-mouth disease, swine vesicular disease, porcine enterovirus infection, duck viral hepatitis, avian encephalomyelitis, encephalomyocarditis, vesicular exanthema of swine, feline calicivirus disease, rabbit viral hemorrhagic disease, canine hepatitis E, Getah virus disease, Japanese encephalitis B/epidemic encephalitis B, forest encephalitis, duck flavivirus infection, swine fever, bovine viral diarrhea-mucosal disease and border disease; and
more preferably, the disease is feline infectious peritonitis.
